(19) United States
(12) Patent Application Publication
(10) Pub. No.: US 2024/0425594 A1
(43) Pub. Date: Dec. 26, 2024

(54) NOVEL PD-1 BINDING DOMAINS

(71) Applicants: MERUS N.V., Utrecht (NL); INCYTE CORPORATION, Wilmington, DE (US)

(72) Inventors: Simon Edward PLYTE, Utrecht (NL); Patrick MAYES, Wilmington, DE (US); Horacio G. NASTRI, Wilmington, DE (US); Shaun M. STEWART, Wilmington, DE (US); Rebecca A. BUONPANE, Wilmington, DE (US)

(73) Assignees: MERUS N.V., Utrecht (NL); INCYTE CORPORATION, Wilmington, DE (US)

(21) Appl. No.: 18/625,703

(22) Filed: Apr. 3, 2024

Related U.S. Application Data
(62) Division of application No. 17/710,243, filed on Mar. 31, 2022, now Pat. No. 11,993,654.

(30) Foreign Application Priority Data
Mar. 31, 2021 (NL) 2027892

(51) Int. Cl. C07K 16/28

(52) U.S. Cl. CPC C07K 16/2818; C07K 2317/31; C07K 2317/35; C07K 2317/522; C07K 2317/524; C07K 2317/526; C07K 2317/53; C07K 2317/565; C07K 2317/76; C07K 2317/92

(57) ABSTRACT

The present disclosure relates to novel PD-1 binding domains that have a higher binding affinity for human PD-1 than a reference PD-1 binding domain. The PD-1 binding domains of the present disclosure further provide a comparable, or equal or higher, potency in blocking ligand binding to human PD-1 than a reference PD-1 antibody. The present disclosure further relates to binding moieties comprising such PD-1 binding domains. Also provided is a method for treating a disease, in particular a disease associated with a suppressed immune system, such as cancer, with a PD-1 binding domain or binding moiety of the present disclosure. The present disclosure further relates to nucleic acids encoding the heavy chain variable region of the PD-1 binding domains, and a vector and cell comprising such nucleic acid.

### SEQUENCE LISTING

A sequence listing in electronic (XML file) format is filed with this application and incorporated herein by reference. The name of the XML file is “AttachB4_2024_0526_SequenceListing.xml”; the file was created on Apr. 3, 2024; the size of the file is 90,298 bytes.

### FIELD

The present disclosure relates to the field of antibodies. In particular it relates to the field of therapeutic antibodies for the treatment of diseases involving aberrant cells. More in particular it relates to novel binding domains that bind to human PD-1, and to binding moieties comprising such binding domain.

### BACKGROUND

Cancer is still a major cause of death in the world, in spite of the many advances that have been made in the treatment of the disease and the increased knowledge of the molecular events that lead to cancer. Traditionally, most cancer drug discovery has focused on agents that block essential cell functions and kill dividing cells. However, in cases of advanced cancer, no matter how aggressively applied, even to the point where patients suffer life-threatening side-effects from the treatment, chemotherapy rarely results in a complete cure. In most cases the tumors in the patients stop growing or temporarily shrink (referred to as remission) only to start proliferating again, sometimes more rapidly (referred to as relapse), and become increasingly more difficult to treat. Over the past years, the focus of cancer drug development has moved away from broadly cytotoxic chemotherapy to targeted cytostatic therapies with less toxicity. Treatment of advanced cancer with targeted therapies has been validated clinically in leukemia and some other cancers. However, in a majority of carcinomas, targeted approaches are still proving not effective enough to completely abolish cancer in the majority of the patients.

Targeting of cancers has been achieved using a variety of different methods including for instance small molecules directed towards signaling proteins on which the cancer depends for survival and/or growth; vaccines with tumor specific proteins; cell therapies with immune cells that actively kill tumor cells, and antibodies that target cytotoxic molecules to the tumor; interfere with signaling and/or that (re)direct the immune system of the host to the tumor cells.

Immune checkpoint proteins, like for instance PD-1, PD-L1, CTLA-4, LAG-3, and TIM-3, are an interesting target for antibody therapy. To date, a number of monospecific antibodies targeting PD-1 have been described, as well as certain bispecific antibodies comprising a PD-1 targeting binding domain. However, each of these antibodies has its own challenges in the production of an effective therapeutic drug. There thus remains a need for the development of novel PD-1 binding domains in order to produce effective PD-1 targeting therapeutic antibodies.

### SUMMARY

One of the objects of the present disclosure is to provide a new pharmaceutical agent for the treatment of human disease, in particular for the treatment of cancer. This object is met by the provision of novel anti-human PD-1 binding domains, and in particular by binding moieties, such as antibodies, comprising such anti-human PD-1 binding domains.

In certain embodiments, the present disclosure provides an anti-human PD-1 binding domain having higher binding affinity for human PD-1 than a reference anti-human PD-1 binding domain, wherein the reference anti-human PD-1 binding domain comprises a heavy chain variable region having an amino acid sequence as set forth in SEQ ID NO: 20 and a light chain variable region having an amino acid sequence as set forth in SEQ ID NO: 21.

In certain embodiments, the present disclosure provides an anti-human PD-1 binding domain, which when monovalently present in a bivalent antibody, provides at least comparable, or equal or higher, potency in blocking ligand binding to PD-1 than a reference anti-human PD-1 antibody, wherein the reference anti-human PD-1 antibody comprises two heavy chain variable regions having an amino acid sequence as set forth in SEQ ID NO: 20 and two light chain variable regions having an amino acid sequence as set forth in SEQ ID NO: 21.

In certain embodiment, the present disclosure provides a binding moiety comprising a PD-1 binding domain as described herein, and in particular a monospecific or multispecific binding moiety, such as a monospecific or multispecific antibody, comprising such PD-1 binding domain.

In certain embodiment, the present disclosure provides a pharmaceutical composition comprising an effective amount of an anti-human PD-1 binding domain or binding moiety as described herein.

In certain embodiments, the present disclosure provides for a PD-1 binding domain, binding moiety comprising the PD-1 binding domain, and pharmaceutical composition as described herein, for use (a) in therapy, or (b) in the treatment of a disease associated with a suppressed immune system, or c) in the treatment of cancer.

In certain embodiments, the present disclosure provides a method for treating a disease, comprising administering an effective amount of a PD-1 binding domain, a binding moiety comprising a PD-1 binding domain, or pharmaceutical composition, as described herein, to an individual in need thereof.

In certain embodiments, the present disclosure provides a method for treating cancer, comprising administering an effective amount of a PD-1 binding domain, a binding moiety comprising a PD-1 binding domain, or pharmaceutical composition, as described herein, to an individual in need thereof.

In certain embodiments, the present disclosure provides a nucleic acid sequence encoding the heavy chain variable region of an anti-human PD-1 binding domain as described herein, a vector and cell comprising such nucleic acid sequence, and a cell producing an anti-human PD-1 binding domain, or the binding moiety, as described herein.

In certain embodiments, the present disclosure provides a method for producing an anti-human PD-1 binding domain as described herein, as well as a method for producing variants thereof.

### DETAILED DESCRIPTION

In certain embodiments, the present disclosure provides several anti-human PD-1 binding domains, the heavy chain variable region having an amino acid sequence as set forth in SEQ ID NO: 1-9. In certain embodiments, the anti-human PD-1 binding domain comprises a heavy chain variable region, wherein the heavy chain variable region comprises the heavy chain CDR1 (HCDR1), heavy chain CDR2 (HCDR2), and heavy chain CDR3 (HCDR3) of any one of the heavy chain variable regions having an amino acid sequence as set forth in SEQ ID NO: 1-9.

Programmed Cell Death 1 protein (PD-1) is a cell surface receptor that belongs to the CD28 family of receptors and is expressed on T cells and pro-B cells. PD-1 is presently known to bind two ligands, PD-L1 and PD-L2. PD-1, functioning as an immune checkpoint, plays an important role in down regulating the immune system by inhibiting the activation of T-cells, which in turn reduces autoimmunity and promotes self-tolerance. The inhibitory effect of PD-1 is thought to be accomplished through a dual mechanism of promoting apoptosis (programmed cell death) in antigen specific T-cells in lymph nodes while simultaneously reducing apoptosis in regulatory T cells (suppressor T cells). PD-1 is also known under a number of different aliases such as PDCD1; Programmed Cell Death 1; Systemic Lupus Erythematosus Susceptibility 2; Protein PD-1; HPD-1; PD1; Programmed Cell Death 1 Protein; CD279 Antigen; CD279; HPD-L; HSLE1; SLEB2; and PD-1. External Ids for PD-1 are HGNC: 8760; Entrez Gene: 5133; Ensembl: ENSG00000188389; OMIM: 600244; and UniProtKB: Q15116. New classes of drugs that block the activity of PD-1, the PD-1 inhibitors, activate the immune system to attack tumors and are therefore used with a certain level of success to treat some types of cancer.

In certain embodiments, the anti-human PD-1 binding domain comprises at least a heavy chain variable region and a light chain variable region. The light chain variable region can be any suitable light chain variable region as described further herein. In certain embodiments, the light chain variable region preferably is a light chain variable region of a light chain that is capable of pairing with multiple heavy chains having different epitope specificities. Such light chain is also referred to in the art as a “common light chain”. In certain embodiments, the present disclosure provides an anti-human PD-1 binding domain having higher binding affinity for human PD-1 than a reference anti-human PD-1 binding domain, wherein the reference anti-human PD-1 binding domain comprises a heavy chain variable region having an amino acid sequence as set forth in SEQ ID NO: 20 and a light chain variable region having an amino acid sequence as set forth in SEQ ID NO: 21.

In certain embodiments, the present disclosure provides a bivalent monospecific binding moiety comprising anti-human PD-1 binding domains, wherein the binding moiety has higher binding affinity for human PD-1 than a reference anti-human PD-1 antibody, wherein the reference anti-human PD-1 antibody comprises two heavy chain variable regions having an amino acid sequence as set forth in SEQ ID NO: 20 and two light chain variable regions having an amino acid sequence as set forth in SEQ ID NO: 21, such as monospecific PD-1 antibody nivolumab or a monospecific PD-1 antibody comprising the variable regions of nivolumab.

Determining if an anti-human PD-1 binding domain has a higher binding affinity for human PD-1 than the reference anti-human PD-1 binding domain can be done by measuring the binding affinity of both anti-human PD-1 binding domains in the same type of assay, using the same assay conditions. Thus, in certain embodiments, the binding affinity of the anti-human PD-1 binding domain or of the bivalent monospecific binding moiety, and the binding affinity of the reference anti-human PD-1 binding domain or of the reference anti-human PD-1 antibody, are measured in the same type of assay, using the same assay conditions. In certain embodiments, the assay is an assay that uses surface plasmon resonance (SPR) to measure binding affinity, such as the biosensor system of Biacore®, or Solution Equilibrium Titration (SET) (see Friguet B et al. (1985) J. Immunol Methods; 77 (2):305-319, and Hanel C et al. (2005) Anal Biochem; 339 (1):182-184). The binding affinity values of the PD-1 binding domains or of the bivalent monospecific binding moieties as provided herein are obtained with the method described in Example 3. In brief, Example 3 describes performing SPR using a Biacore 8K instrument at 25° C. Anti-human Fc antibodies are immobilized via amine coupling on flow cells of an S series sensor chip CM5 with immobilization levels of ˜9000 RU. The desired capturing level (100-150 RU) of anti-PD-1 antibodies is achieved by flowing pre-determined concentration of anti-PD-1 antibodies through the active flow cell of each channel for 60 seconds with 10 μL/min flow rate. A PD-1 three-fold serial dilution concentration series (total 7 concentrations, highest at 300 nM) and running buffer is injected for 240 seconds (association time) immediately followed by running buffer for 480 seconds (dissociation time) at a flow rate of 45 μL/min. Surface is regenerated with 30-second injection of 3 M MgCl₂ with 30 μL/min flow rate. Binding kinetics and affinity parameters are obtained from a global fit of the data to 1 to 1 binding model.

Preferably, SPR is performed with the anti-human PD-1 binding domains in an IgG format, measuring the binding affinity of the monovalent interaction with PD-1.

In certain embodiments, the anti-human PD-1 binding domain or the bivalent monospecific binding moiety has at least a ten-fold higher binding affinity for human PD-1 than the reference anti-human PD-1 binding domain or reference anti-human PD-1 antibody, as measured by SPR as described herein. In certain embodiments, the anti-human PD-1 binding domain or the bivalent monospecific binding moiety has a ten to fifty, ten to forty, ten to thirty, or ten to twenty, fold higher binding affinity for human PD-1 than the reference anti-human PD-1 binding domain or reference anti-human PD-1 antibody, as measured by SPR as described herein. In certain embodiments, the anti-human PD-1 binding domain or the bivalent monospecific binding moiety has a ten-fold higher binding affinity for human PD-1 than the reference anti-human PD-1 binding domain or reference anti-human PD-1 antibody, as measured by SPR as described herein.

In certain embodiments, the anti-human PD-1 binding domain or the bivalent monospecific binding moiety has a binding affinity for human PD-1 in a range of about 0.1-1.0 nM, in particular in a range of about 0.3-0.8 nM, more in particular in a range of about. 0.38-0.78 nM, as measured by SPR as described herein. In certain embodiments, the anti-human PD-1 binding domain or the bivalent monospecific binding moiety has a binding affinity for human PD-1 in a range of 0.1-1.0 nM, in particular in a range of 0.3-0.8 nM, more in particular in a range of 0.38-0.78 nM, as measured by SPR as described herein. In certain embodiments, the binding affinity is the binding affinity of a monovalent interaction with PD-1.

In certain embodiments, the binding affinity is measured with both the anti-human PD-1 binding domain of the present disclosure and the reference anti-human PD-1 binding domain in a bivalent monospecific IgG format. In certain embodiments, the binding affinity is measured with both the anti-human PD-1 binding domain of the present disclosure and the reference anti-human PD-1 binding domain in a bivalent bispecific IgG format. In certain embodiments, the binding affinity is measured with the anti-human PD-1 binding domain of the present disclosure in a bivalent bispecific IgG format and the reference anti-human PD-1 binding domain in a bivalent monospecific IgG format. A bivalent bispecific IgG format may for instance comprise a PD-1 binding domain of the present disclosure, or a reference anti-human PD-1 binding domain, and a binding domain that binds an arbitrarily selected, unrelated target.

The present disclosure also provides an anti-human PD-1 binding domain, which when monovalently present in a bivalent antibody, provides at least comparable, or equal or higher, potency in blocking ligand binding to PD-1 than a reference anti-human PD-1 antibody, wherein the reference anti-human PD-1 antibody comprises two heavy chain variable regions having an amino acid sequence as set forth in SEQ ID NO: 20 and two light chain variable regions having an amino acid sequence as set forth in SEQ ID NO: 21.

The present disclosure also provides a bivalent monospecific anti-human PD-1 binding moiety having at least comparable, or equal or higher, potency in blocking ligand binding to PD-1 than a reference anti-human PD-1 antibody, wherein the reference anti-human PD-1 antibody comprises two heavy chain variable regions having an amino acid sequence as set forth in SEQ ID NO: 20 and two light chain variable regions having an amino acid sequence as set forth in SEQ ID NO: 21.

Determining if an anti-human PD-1 binding domain or bivalent monospecific anti-human PD-1 binding moiety provides a comparable, or equal or higher, potency in blocking ligand binding to PD-1 than the reference anti-human PD-1 antibody can be done by measuring the potency of both the anti-human PD-1 binding domain or moiety and the reference anti-human PD-1 antibody in the same type of assay, using the same assay conditions. Thus, in certain embodiments, the potency in blocking ligand binding to PD-1 of the anti-human PD-1 binding domain or of the bivalent monospecific binding moiety, and the potency in blocking ligand binding to PD-1 of the reference anti-human PD-1 antibody, are measured in the same type of assay, using the same assay conditions. In certain embodiments, the assay is a PD-1/PD-L1 reporter assay. The potency data of the PD-1 binding domains or of the binding moieties comprising the PD-1 binding domains provided herein is obtained with the PD-1/PD-L1 reporter assay as described in Example 2.

In brief, the PD-1/PD-L1 reporter assay described in Example 2 is performed using PD-L1 aAPC/CHO-K1 cells, which are CHO-K1 cells expressing human PD-L1 and an engineered cell surface protein designed to activate cognate TCRs in an antigen-independent manner, and Jurkat T cells expressing human PD-1 and a luciferase reporter driven by an NFAT response element (NFAT-RE). Assay plates comprising the PD-L1 cells or PBS are incubated overnight at 37° C., 5% CO₂ and 95% Relative Humidity. After incubation, wells are emptied and test and control IgG added in serial dilution, starting with 10 μg/ml and performing 6-step 4-fold titration. A basal control, which is control without IgG is also prepared. IgGs of which activities need to be compared directly are incubated on same plate. Jurkat T cells are added, and assay plates are incubated for 6 hours at 37° C., 5% CO₂ and 95% Relative Humidity. Following 6 hours of incubation, plates are left at room temperature for 10 min, and luciferase activity is measured.

Preferably, the anti-human PD-1 binding domain of the present disclosure and the reference anti-human PD-1 antibody are used at the same concentration, preferably both in bivalent monospecific IgG format.

In certain embodiments, a comparable potency in PD-1-ligand blocking activity is a potency within a 5 fold range of the potency in blocking ligand binding to PD-1 of the reference anti-human PD-1 binding domain or anti-human PD-1 binding moiety, and includes a 5, 4, 3, and 2 fold, preferably a 3 fold, deviation, from the potency in blocking ligand binding to PD-1 of the reference anti-human PD-1 antibody.

In certain embodiments, a higher potency in PD-1-ligand blocking activity is a potency that is a 5, 4, 3, or 2 fold, preferably a 3 fold, higher potency than the potency in blocking ligand binding to PD-1 of the reference anti-human PD-1 antibody. In certain embodiments, a higher potency in PD-1-ligand blocking activity is a potency that is a 1.1-2.0 fold, preferably a 1.2-1.8 or 1.2-1.6 fold, more preferably a 1.2-1.4 fold, higher potency than the potency in blocking ligand binding to PD-1 of the reference anti-human PD-1 antibody.

The reference anti-human PD-1 binding domain is the PD-1 binding domain of a nivolumab analog antibody, preferably produced using the same production method as the anti-human PD-1 binding domain subject to comparison. The reference anti-human PD-1 antibody is a nivolumab analog antibody, preferably produced using the same production method as the bivalent monospecific binding moiety comprising an anti-human PD-1 binding domain subject to comparison. A nivolumab analog antibody has the same heavy chain variable region sequence (SEQ ID NO: 20) as nivolumab. A nivolumab analog antibody has the same light chain variable region sequence (SEQ ID NO: 21) as nivolumab.

In certain embodiments, the anti-human PD-1 binding domain comprises a heavy chain variable region, wherein the heavy chain variable region comprises the heavy chain CDR1 (HCDR1), heavy chain CDR2 (HCDR2), and heavy chain CDR3 (HCDR3) of one of the heavy chain variable regions having an amino acid sequence as set forth in SEQ ID NO: 1-9.

CDR sequences can be defined using different methods, including, but not limited to, according to the Kabat numbering scheme (Kabat et al., J. Biol. Chem. 252:6609-6616(1977); and/or Kabat et al., U.S. Dept. of Health and Human Services, “Sequences of proteins of immunological interest” (1991)), the Chothia numbering scheme (Chothia et al., J. Mol. Biol. 196:901-917 (1987); Chothia et al., Nature 342: 877-883, 1989; and/or Al-Lazikani B. et al., J. Mol. Biol., 273: 927-948 (1997)), the numbering system of Honegger and Plukthun (Honegger and Plückthun, J. Mol. Biol., 309:657-670 (2001)), the numbering system of MacCallum (MacCallum et al., J. Mol. Biol. 262:732-745 (1996); and/or Abhinandan and Martin, Mol. Immunol., 45:3832-3839 (2008)), the numbering system of Lefranc (Lefranc M.P. et al., Dev. Comp. Immunol., 27: 55-77 (2003); and/or Honegger and Plückthun, J. Mol. Biol., 309:657-670 (2001)), or according to IMGT (discussed in Giudicelli et al., Nucleic Acids Res. 25: 206-21 1 (1997)).

Each of these numbering schemes base their definition of CDRs on a predicted contribution of amino acid residues in the heavy or light chain variable region to antigen binding. Hence, each method to identify CDRs can be used to identify the CDRs of the binding domains of the present disclosure. In certain embodiments, the heavy chain CDRs of a binding domain of the present disclosure is according to Kabat, Chothia, or IMGT. In certain embodiments, the heavy chain CDRs of a binding domain of the present disclosure is according to Kabat. In certain embodiments, the heavy chain CDRs of a binding domain of the present disclosure is according to Chothia. In certain embodiments, the heavy chain CDRs of a binding domain of the present disclosure is according to IMGT.

In certain embodiments, the anti-human PD-1 binding domain comprises a heavy chain variable region, wherein the heavy chain variable region comprises a heavy chain CDR1 (HCDR1) from a heavy chain variable region having an amino acid sequence from the group consisting of SEQ ID NO: 1-9; a heavy chain CDR2 (HCDR2) from a heavy chain variable region having an amino acid sequence from the group consisting of SEQ ID NO: 1-9; and a heavy chain CDR3 (HCDR3) from a heavy chain variable region having an amino acid sequence from the group consisting of SEQ ID NO: 1-9.

The HCDRs according to Kabat are indicated in bold and underlined in the list of sequences provided herein.

In certain embodiments, the heavy chain variable region of the anti-human PD-1 binding domain comprises:
heavy chain CDR1 (HCDR1), heavy chain CDR2 (HCDR2), and heavy chain CDR3 (HCDR3), having an amino acid sequence as set forth in SEQ ID NO: 22, SEQ ID NO: 23, and SEQ ID NO: 24, respectively;
heavy chain CDR1 (HCDR1), heavy chain CDR2 (HCDR2), and heavy chain CDR3 (HCDR3), having an amino acid sequence as set forth in SEQ ID NO: 25, SEQ ID NO: 26, and SEQ ID NO: 27, respectively;
heavy chain CDR1 (HCDR1), heavy chain CDR2 (HCDR2), and heavy chain CDR3 (HCDR3), having an amino acid sequence as set forth in SEQ ID NO: 28, SEQ ID NO: 29, and SEQ ID NO: 30, respectively;
heavy chain CDR1 (HCDR1), heavy chain CDR2 (HCDR2), and heavy chain CDR3 (HCDR3), having an amino acid sequence as set forth in SEQ ID NO: 31, SEQ ID NO: 32, and SEQ ID NO: 33, respectively;
heavy chain CDR1 (HCDR1), heavy chain CDR2 (HCDR2), and heavy chain CDR3 (HCDR3), having an amino acid sequence as set forth in SEQ ID NO: 34, SEQ ID NO: 35, and SEQ ID NO: 36, respectively;
heavy chain CDR1 (HCDR1), heavy chain CDR2 (HCDR2), and heavy chain CDR3 (HCDR3), having an amino acid sequence as set forth in SEQ ID NO: 37, SEQ ID NO: 38, and SEQ ID NO: 39, respectively;
heavy chain CDR1 (HCDR1), heavy chain CDR2 (HCDR2), and heavy chain CDR3 (HCDR3), having an amino acid sequence as set forth in SEQ ID NO: 40, SEQ ID NO: 41, and SEQ ID NO: 42, respectively;
heavy chain CDR1 (HCDR1), heavy chain CDR2 (HCDR2), and heavy chain CDR3 (HCDR3), having an amino acid sequence as set forth in SEQ ID NO: 43, SEQ ID NO: 44, and SEQ ID NO: 45, respectively; or
heavy chain CDR1 (HCDR1), heavy chain CDR2 (HCDR2), and heavy chain CDR3 (HCDR3), having an amino acid sequence as set forth in SEQ ID NO: 46, SEQ ID NO: 47, and SEQ ID NO: 48, respectively;
wherein each of the HCDRs may comprise at most three, two, or one amino acid substitution.

In certain embodiments, the heavy chain variable region of the anti-human PD-1 binding domain comprises:
heavy chain CDR1 (HCDR1), heavy chain CDR2 (HCDR2), and heavy chain CDR3 (HCDR3), having an amino acid sequence as set forth in SEQ ID NO: 22, SEQ ID NO: 23, and SEQ ID NO: 24, respectively;
heavy chain CDR1 (HCDR1), heavy chain CDR2 (HCDR2), and heavy chain CDR3 (HCDR3), having an amino acid sequence as set forth in SEQ ID NO: 25, SEQ ID NO: 26, and SEQ ID NO: 27, respectively;
heavy chain CDR1 (HCDR1), heavy chain CDR2 (HCDR2), and heavy chain CDR3 (HCDR3), having an amino acid sequence as set forth in SEQ ID NO: 28, SEQ ID NO: 29, and SEQ ID NO: 30, respectively;
heavy chain CDR1 (HCDR1), heavy chain CDR2 (HCDR2), and heavy chain CDR3 (HCDR3), having an amino acid sequence as set forth in SEQ ID NO: 31, SEQ ID NO: 32, and SEQ ID NO: 33, respectively;
heavy chain CDR1 (HCDR1), heavy chain CDR2 (HCDR2), and heavy chain CDR3 (HCDR3), having an amino acid sequence as set forth in SEQ ID NO: 34, SEQ ID NO: 35, and SEQ ID NO: 36, respectively;
heavy chain CDR1 (HCDR1), heavy chain CDR2 (HCDR2), and heavy chain CDR3 (HCDR3), having an amino acid sequence as set forth in SEQ ID NO: 37, SEQ ID NO: 38, and SEQ ID NO: 39, respectively;
heavy chain CDR1 (HCDR1), heavy chain CDR2 (HCDR2), and heavy chain CDR3 (HCDR3), having an amino acid sequence as set forth in SEQ ID NO: 40, SEQ ID NO: 41, and SEQ ID NO: 42, respectively;
heavy chain CDR1 (HCDR1), heavy chain CDR2 (HCDR2), and heavy chain CDR3 (HCDR3), having an amino acid sequence as set forth in SEQ ID NO: 43, SEQ ID NO: 44, and SEQ ID NO: 45, respectively; or
heavy chain CDR1 (HCDR1), heavy chain CDR2 (HCDR2), and heavy chain CDR3 (HCDR3), having an amino acid sequence as set forth in SEQ ID NO: 46, SEQ ID NO: 47, and SEQ ID NO: 48, respectively.

In certain embodiments, a PD-1 binding domain of the present disclosure also includes PD-1 binding domain variants, wherein each of the HCDRs may comprise at most three, two, or one amino acid substitution. In certain embodiments, only one or two HCDRs may comprise at most three, two, or one amino acid substitution.

For example, suitable positions for introducing an amino acid variation include, but are not limited to, the first, second, and/or fourth amino acid of HCDR1; the third, seventh, eighth, ninth, tenth, eleventh, thirteenth, fourteenth, and/or sixteenth amino acid of HCDR2; and/or the sixth and/or thirteenth amino acid of HCDR3. CDR sequences according to Kabat are indicated in bold and underlined in the list of sequences provided herein.

In certain embodiments, the present disclosure thus also provides an anti-human PD-1 binding domain comprising:
HCDR1 having amino acid sequence X₁X₂FX₃S, wherein
   X₁ can be F, Y, T, or H;
   X₂ can be Y, Q, E, H, or D;
   X₃ can be W, or Y; and/or
HCDR2 having amino acid sequence YIX₁YSGX₂X₃X₄X₅X₆PX₇X₈KX₉, wherein
   X₁ can be Y, V, or I;
   X₂ can be S, or G;
   X₃ can be T, Y, S, H, N, W, L, or Q;
   X₄ can be S, or N;
   X₅ can be F, V, or L;
   X₆ can be N, or S;
   X₇ can be S or A;
   X₈ can be F or L;
   X₉ can be S, T, G, D, R, or N; and/or
HCDR3 having amino acid sequence GGYTGX₁GGDWFDX₂, wherein
   X₁ can be Y, H, V, or A;
   X₂ can be P, V, Y, W, F, T, Q, H, or S.

Other suitable positions for introducing an amino acid variation include, but are not limited to, the second, third, fourth, and/or fifth amino acid of HCDR1; the third, fourth, fifth, sixth, eighth, ninth, tenth, eleventh, twelfth, thirteenth, fourteenth, fifteenth, sixteenth and/or seventeenth amino acid of HCDR2; and/or the first, second, sixth, seventh, ninth, tenth, fourteenth, fifteenth, sixteenth and/or eighteenth amino acid of HCDR3. CDR sequences according to Kabat are indicated in bold and underlined in the list of sequences provided herein.

In certain embodiments, the present disclosure thus also provides an anti-human PD-1 binding domain comprising:
HCDR1 having amino acid sequence RX₁X₂X₃X₄, wherein
   X₁ can be F, or Y;
   X₂ can be T, A, or V;
   X₃ can be M, L, or V;
   X₄ can be S, H, N, V, or T; and/or
HCDR2 having amino acid sequence WIX₁X₂X₃X₄GX₅X₆X₇X₈X₉X₁₀X₁₁X₁₂X₁₃X₁₄, wherein
   X₁ can be N, or D;
   X₂ can be P, S, or T;
   X₃ can be N, or Q;
   X₄ can be T, or D;
   X₅ can be N, S, T, K, L, or E;
   X₆ can be P, Y, A, H, or F;
   X₇ can be T, or S;
   X₈ can be Y, F, or H;
   X₉ can be A, G, V, or F;
   X₁₀ can be Q, R, N, L, T, or S;
   X₁₁ can be D, A, G, or S;
   X₁₂ can be F, V, or A;
   X₁₃ can be T, K, H, G;
   X₁₄ can be G, N, E, or D; and/or
HCDR3 having amino acid sequence X₁X₂GYCX₃X₄DX₅CYPNX₆X₇X₈DX₉, wherein
   X₁ can be I, S, or V;
   X₂ can be L, Q, or N;
   X₃ can be N, G, S, or D;
   X₄ can be T, S, P, N, or E;
   X₅ can be N, or I;
   X₆ can be W, G, Q, H, W, A, or L;
   X₇ can be I, V, or L;
   X₈ can be F, L, or I;
   X₉ can be Y, S, N, I, R, H, V, T, K, A, or L.

In certain embodiments, the anti-human PD-1 binding domain of the present disclosure comprises a heavy chain variable region having an amino acid sequence as set forth in any one of SEQ ID NO: 1-9, or having at least 80%, preferably 85%, more preferably 90%, or most preferably 95% sequence identity thereto.

In certain embodiments, a PD-1 binding domain of the present disclosure also includes PD-1 binding domain variants, which, in addition to the variations in the HCDRs referred to above, comprise one or more variations in the framework regions. In certain embodiments, a PD-1 binding domain variant of the present disclosure comprises no variations in the CDR regions but comprises one or more variations in the framework regions. Such variants have at least 80%, preferably 85%, more preferably 90%, or most preferably 95% sequence identity to the sequences disclosed herein, and are expected to retain PD-1 binding specificity. Thus, in certain embodiments, a PD-1 binding domain of the present disclosure comprises:
a heavy chain variable region having at least 80%, preferably 85%, more preferably 90%, or most preferably 95% sequence identity to the amino acid sequence as set forth in SEQ ID NO: 1, which heavy chain variable region comprises a HCDR1 amino acid sequence as set forth in SEQ ID NO: 25; a HCDR2 amino acid sequence as set forth in SEQ ID NO: 26; and a HCDR3 amino acid sequence as set forth in SEQ ID NO: 27;
a heavy chain variable region having at least 80%, preferably 85%, more preferably 90%, or most preferably 95% sequence identity to the amino acid sequence as set forth in SEQ ID NO: 2, which heavy chain variable region comprises a HCDR1 amino acid sequence as set forth in SEQ ID NO: 28; a HCDR2 amino acid sequence as set forth in SEQ ID NO: 29; and a HCDR3 amino acid sequence as set forth in SEQ ID NO: 30;
a heavy chain variable region having at least 80%, preferably 85%, more preferably 90%, or most preferably 95% sequence identity to the amino acid sequence as set forth in SEQ ID NO: 3, which heavy chain variable region comprises a HCDR1 amino acid sequence as set forth in SEQ ID NO: 31; a HCDR2 amino acid sequence as set forth in SEQ ID NO: 32; and a HCDR3 amino acid sequence as set forth in SEQ ID NO: 33;
a heavy chain variable region having at least 80%, preferably 85%, more preferably 90%, or most preferably 95% sequence identity to the amino acid sequence as set forth in SEQ ID NO: 4, which heavy chain variable region comprises a HCDR1 amino acid sequence as set forth in SEQ ID NO: 34; a HCDR2 amino acid sequence as set forth in SEQ ID NO: 35; and a HCDR3 amino acid sequence as set forth in SEQ ID NO: 36;
a heavy chain variable region having at least 80%, preferably 85%, more preferably 90%, or most preferably 95% sequence identity to the amino acid sequence as set forth in SEQ ID NO: 5, which heavy chain variable region comprises a HCDR1 amino acid sequence as set forth in SEQ ID NO: 37; a HCDR2 amino acid sequence as set forth in SEQ ID NO: 38; and a HCDR3 amino acid sequence as set forth in SEQ ID NO: 39;
a heavy chain variable region having at least 80%, preferably 85%, more preferably 90%, or most preferably 95% sequence identity to the amino acid sequence as set forth in SEQ ID NO: 6, which heavy chain variable region comprises a HCDR1 amino acid sequence as set forth in SEQ ID NO: 40; a HCDR2 amino acid sequence as set forth in SEQ ID NO: 41; and a HCDR3 amino acid sequence as set forth in SEQ ID NO: 42;
a heavy chain variable region having at least 80%, preferably 85%, more preferably 90%, or most preferably 95% sequence identity to the amino acid sequence as set forth in SEQ ID NO: 7, which heavy chain variable region comprises a HCDR1 amino acid sequence as set forth in SEQ ID NO: 43; a HCDR2 amino acid sequence as set forth in SEQ ID NO: 44; and a HCDR3 amino acid sequence as set forth in SEQ ID NO: 45;
a heavy chain variable region having at least 80%, preferably 85%, more preferably 90%, or most preferably 95% sequence identity to the amino acid sequence as set forth in SEQ ID NO: 8, which heavy chain variable region comprises a HCDR1 amino acid sequence as set forth in SEQ ID NO: 46; a HCDR2 amino acid sequence as set forth in SEQ ID NO: 47; and a HCDR3 amino acid sequence as set forth in SEQ ID NO: 48; or
a heavy chain variable region having at least 80%, preferably 85%, more preferably 90%, or most preferably 95% sequence identity to the amino acid sequence as set forth in SEQ ID NO: 9, which heavy chain variable region comprises a HCDR1 amino acid sequence as set forth in SEQ ID NO: 22; a HCDR2 amino acid sequence as set forth in SEQ ID NO: 23; and a HCDR3 amino acid sequence as set forth in SEQ ID NO: 24.

In certain embodiments, a PD-1 binding domain of the present disclosure comprises a light chain variable region. An example of a suitable light chain variable region is a light chain variable region comprising a light chain CDR1 (LCDR1), light chain CDR2 (LCDR2), and light chain CDR3 (LCDR3), having an amino acid sequence as set forth in SEQ ID NO: 49, SEQ ID NO: 50, and SEQ ID NO: 51, respectively, wherein each of the LCDRs may comprise at most three, two, or one amino acid substitution. In certain embodiments, a suitable light chain variable region is a light chain variable region comprising a light chain CDR1 (LCDR1), light chain CDR2 (LCDR2), and light chain CDR3 (LCDR3), having an amino acid sequence as set forth in SEQ ID NO: 49, SEQ ID NO: 50, and SEQ ID NO: 51, respectively. In certain embodiments, such light chain variable region may comprise a light chain variable region having an amino acid sequence as set forth in SEQ ID NO: 16, or having at least 80%, preferably 85%, more preferably 90%, or most preferably 95% sequence identity thereto. A light chain or light chain variable region comprising these LCDRs and/or light chain variable region is the light chain referred to in the art as VK1-39/JK1. This is a common light chain.

In certain embodiments, a PD-1 binding domain of the present disclosure comprises a light chain variable region having at least 80%, preferably 85%, more preferably 90%, or most preferably 95% sequence identity to the amino acid sequence as set forth in SEQ ID NO: 16, which light chain variable region comprises a LCDR1 amino acid sequence as set forth in SEQ ID NO: 49; a LCDR2 amino acid sequence as set forth in SEQ ID NO: 50; and a LCDR3 amino acid sequence as set forth in SEQ ID NO: 51.

The term ‘common light chain’ according to the invention refers to a light chain that is capable of pairing with multiple different heavy chains, i.e. heavy chains having different antigen or epitope binding specificities. A common light chain is particularly useful in the generation of, for instance, bispecific antibodies, where antibody production is more efficient when both binding domains comprise the same light chain. The term “common light chain” encompasses light chains that are identical or have some amino acid sequence differences while the binding specificity of the full length antibody is not affected. It is for instance possible within the scope of the definition of common light chains as used herein, to prepare or find light chains that are not identical but still functionally equivalent, e.g., by introducing and testing conservative amino acid changes, changes of amino acids in regions that do not or only partly contribute to binding specificity when paired with the heavy chain, and the like.

Apart from a common light chain comprising the LCDRs and/or light chain variable region referred to above, other common light chains known in the art may be used. Examples of such common light chains include, but are not limited to: VK1-39/JK5, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 52. The LCDRs according to IMGT are indicated in bold and underlined therein. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 52, wherein each of the LCDRs may comprise at most three, two, or one amino acid substitution. In certain embodiments, the light chain comprises a light chain variable region having an amino acid sequence as set forth in SEQ ID NO: 52, or having at least 80%, preferably 85%, more preferably 90%, or most preferably 95% sequence identity thereto; VK3-15/JK1, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 53. The LCDRs according to IMGT are indicated in bold and underlined therein. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 53, wherein each of the LCDRs may comprise at most three, two, or one amino acid substitution. In certain embodiments, the light chain comprises a light chain variable region having an amino acid sequence as set forth in SEQ ID NO: 53, or having at least 80%, preferably 85%, more preferably 90%, or most preferably 95% sequence identity thereto; VK3-20/JK1, comprising a light chain variable region comprising a light chain CDR1 (LCDR1), light chain CDR2(LCDR2), and light chain CDR3 (LCDR3), of a light chain variable region having an amino acid sequence as set forth in SEQ ID NO: 54. The LCDRs according to IMGT are indicated in bold and underlined therein. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 54, wherein each of the LCDRs may comprise at most three, two, or one amino acid substitution. In certain embodiments, the light chain comprises a light chain variable region having an amino acid sequence as set forth in SEQ ID NO: 54, or having at least 80%, preferably 85%, more preferably 90%, or most preferably 95% sequence identity thereto; and VL3-21/JL3, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 55. The LCDRs according to IMGT are indicated in bold and underlined therein. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 55, wherein each of the LCDRs may comprise at most three, two, or one amino acid substitution. In certain embodiments, the light chain comprises a light chain variable region having an amino acid sequence as set forth in SEQ ID NO: 55, or having at least 80%, preferably 85%, more preferably 90%, or most preferably 95% sequence identity thereto.

VK1-39 is short for Immunoglobulin Variable Kappa 1-39 Gene. The gene is also known as Immunoglobulin Kappa Variable 1-39; IGKV139; IGKV1-39; IgVK1-39. External Ids for the gene are HGNC: 5740; Entrez Gene: 28930; Ensembl: ENSG00000242371. A preferred amino acid sequence for VK1-39 is given as SEQ ID NO: 56. This is the sequence of the V-region. The V-region can be combined with one of five J-regions. Two preferred joined sequences are indicated as VK1-39/JK1 and VK1-39/JK5; alternative names are IgVκ1-39*01/IGJκ1*01 or IgVκ1-39*01/IGJK5*01 (nomenclature according to the IMGT database worldwide web at imgt.org). These names are exemplary and encompass allelic variants of the gene segments.

VK3-15 is short for Immunoglobulin Variable Kappa 3-15 Gene. The gene is also known as Immunoglobulin Kappa Variable 3-15; IGKV315; IGKV3-15; IgVκ3-15. External Ids for the gene are HGNC: 5816; Entrez Gene: 28913; Ensembl: ENSG00000244437. A preferred amino acid sequence for VK3-15 is given as SEQ ID NO: 57. This is the sequence of the V-region. The V-region can be combined with one of five J-regions. A preferred joined sequence is indicated as VK3-15/JK1; alternative name is Vκ3-15*01/IGJκ1*01 (nomenclature according to the IMGT database worldwide web at imgt.org). This name is exemplary and encompasses allelic variants of the gene segments.

VK3-20 is short for Immunoglobulin Variable Kappa 3-20 Gene. The gene is also known as Immunoglobulin Kappa Variable 3-20; IGKV320; IGKV3-20; IgVκ3-20. External Ids for the gene are HGNC: 5817; Entrez Gene: 28912; Ensembl: ENSG00000239951. A preferred amino acid sequence for VK3-20 is as SEQ ID NO: 58. This is the sequence of the V-region. The V-region can be combined with one of five J-regions. A preferred joined sequence is indicated as VK3-20/JK1; alternative name is IgVκ3-20*01/IGJκ1*01 (nomenclature according to the IMGT database worldwide web at imgt.org). This name is exemplary and encompasses allelic variants of the gene segments.

VL3-21 is short for Immunoglobulin Variable Lambda 3-21 Gene. The gene is also known as Immunoglobulin Lambda Variable 3-21; IGLV321; IGLV3-21; IgVλ3-21. External Ids for the gene are HGNC: 5905; Entrez Gene: 28796; Ensembl: ENSG00000211662.2. A preferred amino acid sequence for VL3-21 is given as SEQ ID NO: 59. This is the sequence of the V-region. The V-region can be combined with one of five J-regions. A preferred joined sequence is indicated as VL3-21/JL3; alternative name is IgVλ3-21/IGJλ3 (nomenclature according to the IMGT database worldwide web at imgt.org). This name is exemplary and encompasses allelic variants of the gene segments.

Further, any light chain variable region of a PD-1 antibody available in the art may be used, or any other light chain variable region that can readily be obtained, such as from, for instance, an antibody display library by showing antigen binding activity when paired with a PD-1 binding domain of the invention.

In certain embodiments, a PD-1 binding domain of the present disclosure may further comprise a CH1 and CL region. Any CH1 domain may be used, in particular a human CH1 domain. An example of a suitable CH1 domain is provided by the amino acid sequence provided as SEQ ID NO: 17. Any CL domain may be used, in particular a human CL. An example of a suitable CL domain is provided by the amino acid sequence provided as SEQ ID NO: 60.

A PD-1 binding domain of the present disclosure can be used as a binding domain in a binding moiety. A “binding moiety” refers to a proteinaceous molecule and includes for instance all antibody formats available in the art, such as for example a full length IgG antibody, immunoconjugates, diabodies, BiTEs, Fab fragments, scFv, tandem scFv, single domain antibody (like V_{HH} and V_{H}), minibodies, scFab, scFv-zipper, nanobodies, DART molecules, TandAb, Fab-scFv, F(ab)′2, F(ab)′2-scFv2, and intrabodies.

In one embodiment, the binding moiety of the present disclosure is a monospecific binding moiety, in particular a monospecific antibody. A monospecific antibody according to the present disclosure is an antibody, in any antibody format, that comprises one or more binding domains with specificity for a single target. In certain embodiments, a monospecific binding moiety of the present disclosure may further comprise an Fc region or a part thereof. In certain embodiments, a monospecific binding moiety of the present disclosure is an IgG1 antibody.

In one embodiment, the binding moiety of the present disclosure is a multispecific binding moiety, in particular a multispecific antibody. A multispecific antibody according to the present disclosure is an antibody, in any antibody format, that comprises at least two binding domains which have specificity for at least two different targets or epitopes. In certain embodiments, a multispecific antibody of the invention is a bispecific antibody. In certain embodiments, a multispecific antibody of the present disclosure may further comprise an Fc region or a part thereof. In certain embodiments, a multispecific antibody of the present disclosure is an IgG1 antibody.

The present disclosure further provides a nucleic acid sequence encoding the heavy chain variable region of an anti-human PD-1 binding domain or binding moiety as described herein.

Further provided herein is a vector useful for producing a PD-1 binding domain or binding moiety of the present disclosure. In certain embodiments, such expression vector comprises a polynucleotide encoding the heavy chain variable region of the anti-human PD-1 binding domain as described herein. In certain embodiments, a vector of the present disclosure may further encode a CH1 region and preferably a hinge, CH2 and CH3 region. In certain embodiments, the vector of the present disclosure may further comprise at least one polynucleotide encoding a light chain variable region, and preferably a CL region. In certain embodiments, the light chain variable region can be a common light chain variable region as described herein.

The present disclosure also provides a cell comprising a nucleic acid sequence encoding the heavy chain variable region of an anti-human PD-1 binding domain as described herein. In certain embodiments, a cell of the present disclosure may further comprises a nucleic acid sequence encoding a CH1 region and preferably a hinge, CH2 and CH3 region. In certain embodiments, a cell of the present disclosure may further comprises at least one nucleic acid sequence encoding a light chain variable region, and preferably a CL region. In certain embodiments, the light chain variable region can be a common light chain variable region as described herein.

Further provided herein is a cell producing an anti-human PD-1 binding domain or a binding moiety as described herein. In certain embodiments, such cell can be a recombinant cell, which has been transformed with a vector of the present disclosure.

Further provided herein is a method for producing an anti-human PD-1 binding domain, or a binding moiety comprising an anti-human PD-1 binding domain, of the present disclosure, wherein the method comprises culturing a cell as described herein and recovering the anti-human PD-1 binding domain, or the binding moiety comprising an anti-human PD-1 binding domain, from the cell or supernatant.

Further provided herein is a method for producing a variant of an anti-human PD-1 binding domain of the present disclosure, wherein the method comprises:
generating a sequence variant of a heavy chain variable region as described herein; and
expressing the sequence variant and a light chain variable region as described herein in a cell.

Methods for generating sequence variants are well known in the art. One can take a random approach in generating sequence variants or a targeted approach, where one can for instance aim at introducing variations that are likely to increase or decrease binding affinity. Routine methods for affinity maturing antibody binding domains are widely known in the art, see for instance Tabasinezhad M. et al. Immunol Lett. 2019;212:106-113. One can also aim at introducing variations that mitigate developability risks with a view on producing a binding domain, or moiety comprising such binding domain, at large scale. Variations may be introduced that are likely not to cause a loss in binding specificity and/or affect binding affinity. Whether amino acid residues within the CDRs and/or framework regions can be substituted, for instance with a conservative amino acid residue, and without, or substantially without, loss in binding specificity and/or affinity, can be determined by methods well known in the art. Experimental examples include, but are not limited to, for instance, alanine scanning (Cunningham BC, Wells JA. Science. 1989;244(4908):1081-5), and deep mutational scanning (Araya CL, Fowler DM. Trends Biotechnol. 2011;29(9):435-42). Computational methods have also been developed that can predict the effect of amino acid variation, such as for instance described in Sruthi CK, Prakash M. PLoS One. 2020;15(1):e0227621, Choi Y. et al. PLoS One. 2012;7(10):e46688, and Munro D, Singh M. Bioinformatics. 2020;36(22-23):5322-9.

Further provided herein are any variant anti-human PD-1 binding domains produced by the above described method; binding moieties, such as antibodies, comprising any of said variant binding domains; a pharmaceutical composition comprising any of said variant anti-human PD-1 binding domains or binding moieties; nucleic acids encoding any of said variant binding domains; vectors and cells comprising said nucleic acids; and use of said variant binding domains or pharmaceutical composition for the treatment of cancer.

### Pharmaceutical Composition and Methods

An anti-human PD-1 binding domain of the present disclosure or a binding moiety of the present disclosure can be used in a pharmaceutical composition, together with a pharmaceutically acceptable carrier, to effectively treat a disease, preferably a disease associated with a suppressed immune system, in particular cancer. Treatment includes the administration of an effective amount of the PD-1 binding domain, binding moiety, or pharmaceutical composition, to a subject in need thereof.

In certain embodiments, the present disclosure provides an anti-human PD-1 binding domain, a binding moiety, or a pharmaceutical composition, as described herein for use in therapy.

In certain embodiments, the present disclosure provides an anti-human PD-1 binding domain, a binding moiety, or a pharmaceutical composition, as described herein for use in the treatment of a disease associated with a suppressed immune system, in particular cancer.

In certain embodiments, the present disclosure provides a method for treating a disease, wherein the method comprises administering an effective amount of an anti-human PD-1 binding domain, a binding moiety, or a pharmaceutical composition as described herein to an individual in need thereof.

In certain embodiments, the present disclosure provide a method for treating a disease associated with a suppressed immune system, in particular cancer, wherein the method comprises administering an effective amount of an anti-human PD-1 binding domain, a binding moiety, or a pharmaceutical composition as described herein to an individual in need thereof.

As used herein, the terms “individual”, “subject” and “patient” are used interchangeably and refer to a mammal such as a human, mouse, rat, hamster, guinea pig, rabbit, cat, dog, monkey, cow, horse, pig and the like (e.g., a patient, such as a human patient, having cancer).

The terms “treat,” “treating,” and “treatment,” as used herein, refer to any type of intervention or process performed on or administering an active agent or combination of active agents to a subject with the objective of curing or improving a disease or symptom thereof. This includes reversing, alleviating, ameliorating, inhibiting, or slowing down a symptom, complication, condition or biochemical indicia associated with a disease, as well as preventing the onset, progression, development, severity or recurrence of a symptom, complication, condition or biochemical indicia associated with a disease.

As used herein, “effective treatment” or “positive therapeutic response” refers to a treatment producing a beneficial effect, e.g., amelioration of at least one symptom of a disease or disorder, e.g., cancer. A beneficial effect can take the form of an improvement over baseline, including an improvement over a measurement or observation made prior to initiation of therapy according to the method. For example, a beneficial effect can take the form of slowing, stabilizing, stopping or reversing the progression of a cancer in a subject at any clinical stage, as evidenced by a decrease or elimination of a clinical or diagnostic symptom of the disease, or of a marker of cancer. Effective treatment may, for example, decrease in tumor size, decrease the presence of circulating tumor cells, reduce or prevent metastases of a tumor, slow or arrest tumor growth and/or prevent or delay tumor recurrence or relapse.

The term “therapeutic amount” or “effective amount” refers to an amount of an agent or combination of agents that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In some embodiments, a therapeutic amount is an amount sufficient to delay tumor development. In some embodiments, a therapeutic amount is an amount sufficient to prevent or delay tumor recurrence.

The effective amount of the agent or composition may: (i) reduce the number of cancer cells; (ii) reduce tumor size; (iii) inhibit, retard, slow to some extent and may stop cancer cell infiltration into peripheral organs; (iv) inhibit tumor metastasis; (v) inhibit tumor growth; (vi) prevent or delay occurrence and/or recurrence of tumor; and/or (vii) relieve to some extent one or more of the symptoms associated with the cancer.

An effective amount may vary according to factors such as the disease state, age, sex, and weight of the individual to be treated, and the ability of the agent or combination of agents to elicit a desired response in the individual.

An effective amount can be administered in one or more administrations.

An effective amount also includes an amount that balances any toxic or detrimental effects of the agent or combination of agents and the therapeutically beneficial effects.

The term “agent” refers to a therapeutically active substance, in the present case a PD-1 binding domain of the present disclosure, a binding moiety of the present disclosure, or a pharmaceutical composition of the present disclosure.

### General Terms

As used herein, “to comprise” and its conjugations is used in its non-limiting sense to mean that items following the word are included, but items not specifically mentioned are not excluded.

The articles “a” and “an” are used herein to refer to one or more of the grammatical object of the article. By way of example, “an element” means one or more elements.

A reference herein to a patent document or other matter is not to be taken as an admission that that document or matter was known or that the information it contains was part of the common general knowledge at the priority date of any of the claims.

All patent and literature references cited in the present specification are hereby incorporated by reference in their entirety.

Note that in the present specification, unless stated otherwise, amino acid positions assigned to CDRs and frameworks in a variable region of an antibody or antibody fragment are specified according to Kabat's numbering (see Sequences of Proteins of Immunological Interest (National Institute of Health, Bethesda, Md., 1987 and 1991)). Amino acids in the constant regions are indicated according to the EU numbering system.

Accession numbers are primarily given to provide a further method of identification of a target, the actual sequence of the protein bound may vary, for instance because of a mutation in the encoding gene such as those occurring in some cancers or the like. The antigen binding site binds the antigen and a variety of variants thereof, such as those expressed by some antigen positive immune or tumor cells.

When herein reference is made to a gene, a protein, the reference is preferably to the human form of the gene or protein. When herein reference is made to a gene or protein reference is made to the natural gene or protein and to variant forms of the gene or protein as can be detected in tumors, cancers and the like, preferably as can be detected in human tumors, cancers and the like.

HGNC stands for the HUGO Gene nomenclature committee. The number following the abbreviation is the accession number with which information on the gene and protein encoded by the gene can be retrieved from the HGNC database. Entrez Gene provides the accession number or gene ID with which information on the gene or protein encoded by the gene can be retrieved from the NCBI (National Center for Biotechnology Information) database. Ensemble provides the accession number with which information on the gene or protein encoded by the gene can be obtained from the Ensemble database. Ensembl is a joint project between EMBL-EBI and the Wellcome Trust Sanger Institute to develop a software system which produces and maintains automatic annotation on selected eukaryotic genomes.

### BRIEF DESCRIPTION OF THE DRAWINGS

In the Figures, bivalent monospecific antibodies are indicated in the format SEQ ID NO: A, where SEQ ID NO: A refers to the heavy chain variable sequence of both binding domains. Each binding domain of the monospecific antibodies comprises a light chain. In the Examples, which are used to illustrate the present disclosure but are not intended to limit the disclosure in any way, each binding domain of the monospecific antibodies comprises a light chain variable region having an amino acid sequence as set forth in SEQ ID NO: 16 and a light chain constant region having an amino acid sequence as set forth in SEQ ID NO: 60. The monospecific antibodies preferably are IgG1 antibodies comprising a CH1, hinge, CH2, and CH3. In the Examples, which are used to illustrate the present disclosure but are not intended to limit the disclosure in any way, monospecific antibodies were screened in IgG1 format, wherein the PD-1 binding heavy chains comprise a CH1 having an amino acid sequence as set forth in SEQ ID NO: 17, a CH2 having an amino acid sequence as set forth in SEQ ID NO: 18, and a CH3 having an amino acid sequence as set forth in SEQ ID NO: 19 .

Bivalent monospecific nivolumab analog antibody is indicated in the format SEQ ID NO: A/SEQ ID NO: B, where SEQ ID NO: A refers to the respective heavy chain sequence and SEQ ID NO: B refers to the respective light chain sequence. This reference antibody analog is used in IgG1 or IgG4 format, and each binding domain comprises a light chain.

Bivalent bispecific antibodies are indicated in the format SEQ ID NO: AxAntigen A, where SEQ ID NO: A refers to the heavy chain variable sequence of the PD-1 binding domain and Antigen A refers to the heavy chain variable sequence of an unrelated, arbitrarily selected antigen. Each binding domain of the bispecific antibodies comprises a light chain. The bispecific antibodies are IgG1 antibodies, comprising a CH1, hinge, CH2, and CH3.

FIG. 1 shows the results of screening of affinity matured variants in a PD-1/PD-L1 reporter assay. A) IgG's comprising affinity matured heavy chain variable regions having an amino acid sequence as set forth in SEQ ID NO: 1, SEQ ID NO: 2, and SEQ ID NO: 6; were compared with parental antibody comprising a heavy chain variable region having an amino acid sequence as set forth in SEQ ID NO: 9, a nivolumab analog (SEQ ID NO: 11/SEQ ID NO: 14) as a positive control, and a negative control (SEQ ID NO: 15/SEQ ID NO: 16). B) IgG's comprising affinity matured heavy chain variable regions having an amino acid sequence as set forth in SEQ ID NO: 3, SEQ ID NO: 4, and SEQ ID NO: 5; were compared with parental antibody comprising a heavy chain variable region having an amino acid sequence as set forth in SEQ ID NO: 10, nivolumab analogs (SEQ ID NO: 11/SEQ ID NO: 14 and SEQ ID NO: 12/SEQ ID NO: 14) as positive controls, and a negative control (SEQ ID NO: 15/SEQ ID NO: 16).

FIG. 2 shows the binding affinity of the PD-1 binding domains comprising a heavy chain variable region having an amino acid sequence as set forth in SEQ ID NO: 1, SEQ ID NO: 6, or SEQ ID NO: 5, in bivalent monospecific format, compared with bivalent monospecific nivolumab analog 1 (SEQ ID NO: 11/SEQ ID NO: 14; in quadruplicate) and the PD-1 binding domain of nivolumab analog 1 as part of a bivalent bispecific antibody (SEQ ID NO: 11/SEQ ID NO: 14 x SEQ ID NO: 15/SEQ ID NO: 16).

FIG. 3 shows the binding affinity of bispecific antibodies comprising SEQ ID NO: 7 and a binding domain against an arbitrarily selected antigen not impacting PD-1 binding tested in the assay, and SEQ ID NO: 8 and a binding domain against an arbitrarily selected antigen not impacting PD-1 binding tested in the assay, to human and cynomolgus PD-1, compared with a nivolumab analog (SEQ ID NO: 13/SEQ ID NO: 14).

The following Examples illustrate the present disclosure but are not intended to limit the disclosure in any way.

### EXAMPLES

### Example 1—Generation of Anti-Human PD-1 Binding Domains

Anti-human PD-1 binding domains can be obtained by methods known in the art, such as for instance as described in WO 2019/009728. A large panel of heavy chain variable regions were obtained by immunizing transgenic mice comprising a common IGKV1-39 light chain (MeMo® mice) with human PD-1 antigenic moieties, including the use of different forms of DNA, protein and cell-based antigen delivery. Heavy chain variable regions of SEQ ID NO: 9 and SEQ ID NO: 10 were selected for affinity maturation. This resulted in 202 affinity matured variants of which a number were selected for further characterization in a PD-1/PD-L1 reporter assay.

### Example 2—Potency of PD-1 IgG

In order to confirm that the affinity matured PD-1 heavy chain variable regions in IgG format are at least as potent as their parental IgG's, affinity matured variants were screened in a PD-1/PD-L1 reporter assay. Also included in the assay were the parental anti-PD-1 IgG's, an anti-PD-1 antibody comprising the heavy chain variable region (SEQ ID NO: 11) and light chain variable region (SEQ ID NO: 14) of nivolumab (Fc-silenced IgG1 nivolumab analog 1), and an anti-PD-1 antibody comprising the heavy chain variable region (SEQ ID NO: 12) and light chain variable region (SEQ ID NO: 14) of nivolumab (IgG4 nivolumab analog 2) as positive controls, and an anti RSV-G antibody comprising the heavy chain variable region having SEQ ID NO: 15 and light chain variable region having SEQ ID NO: 16 as a negative control. The last 2 wells in this column were left without IgG as a basal level control.

The PD-1-PD-L1 reporter assay was performed according to manufacturer's protocol (Promega, cat. no. J1255), which uses two cell lines: PD-L1 aAPC/CHO-K1 cells, which are CHO-K1 cells expressing human PD-L1 and an engineered cell surface protein designed to activate cognate TCRs in an antigen-independent manner (Promega, cat. no. J109A); and PD-1 effector cells: Jurkat T cells expressing human PD-1 and a luciferase reporter driven by an NFAT response element (NFAT-RE) (Promega, cat. no. J115A).

On day 1, Cell Recovery Medium for PD-L1 cells was prepared at room temperature: 10% FBS (Sigma, cat. no. F2442) in DMEM/F12 (Life Technologies, cat. no. 21765). The required number of PD-L1 cell vials (J109A; 1 vial per 32 IgG's to be tested) were removed from the freezer, thawed quickly at 37° C. and cells transferred to a 50 ml tube. Cell Recovery Medium was slowly added to cells, 14.5 ml/vial, volume doubling per minute. Wells of ½-area plates (Corning, cat. no. 3688) were filled with this cell suspension at 50 μl/well or with 50 μl PBS (Invitrogen, cat. no. 10010). Assay plates were incubated overnight at 37° C., 5% CO2 and 95% Relative Humidity.

On the second day, 2× concentrated Assay Buffer was prepared: 4% FBS (Sigma, cat. no. F2442) in RPMI 1640 (Promega kit or Life Technologies, cat. no. 21875) at room temperature. 2× concentrated test and control IgG solutions were prepared in PBS. Serial dilutions of test and control IgG's were also made in PBS in U-bottom plates (Nunc, cat. no. 268152), starting with 10 μg/ml and performing 6-step 4-fold titration. Positive and negative control IgG serial dilutions were prepared in PBS on separate deep well plates (Greiner Bio-one, cat. no. 780270). Basal control, which is control without IgG was also prepared. IgG's of which activities need to be compared directly were incubated on same plate as much as possible, to avoid inter-plate variation.

Assay plates were taken out of the incubator and flicked to empty wells. 20 μl of IgG solution was added to assay plate, starting with transfer of lowest IgG concentration followed by higher concentration with same pipet tips.

Required number of PD-1 effector cells (J115A: 1 vial per 32 IgG's to be tested) were removed from freezer, thawed quickly at 37° C. and gently mixed by pipetting up and down. Cells from all vials were transferred to a 50 ml tube. 2× concentrated Assay Buffer (5.9 ml per vial of cells) was slowly added to cells such that volume doubled per minute. 20 μl of effector cell suspension was added to wells on assay plates. Plates were incubated for 6 hours at 37° C., 5% CO2 and 95% Relative Humidity. Following 6 hours incubation, plates were pre-incubated at room temperature for 10 min.

Luciferase activity was measured using the Bio-Glo™ luciferase Assay System (Promega, cat. no. G7941). Bio-Glo™ Luciferase Assay Buffer (protected from light) was equilibrated to room temperature overnight and thoroughly mixed with Bio-Glo™ Luciferase Assay Substrate. 40 μl of Bio-Glo luciferase was added to each well on the assay plate and luminescence measured after 5-10 min on EnVision plate reader (PerkinElmer, Model 2104-0040A Luminescence mode). Readout was obtained in Relative light unit (RLU) values. Fold Induction which is ratio of experimental activity to control activity was calculated as RLU value of IgG-X/RLU value of no IgG. Fold Induction was plotted against log IgG concentrations and the sigmoid curve fitted in GraphPad Prism using non-linear regression and the log (inhibitor) vs. response (three parameters) equation.

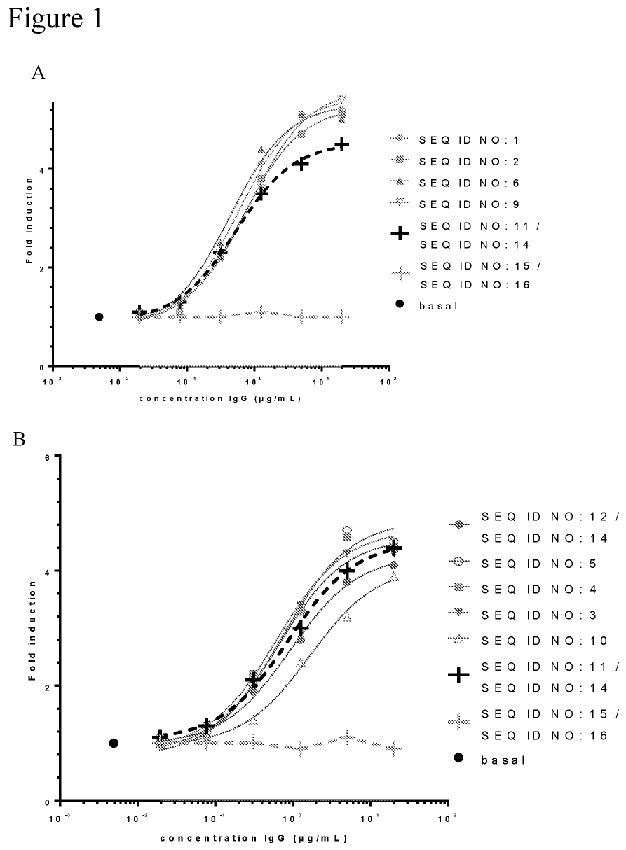

Results are shown in FIG. 1. All controls displayed the expected activities and were consistent in different plates. The affinity matured variants were at least as potent as their parental IgG, and as potent or more potent than nivolumab analog 1. EC50 values of the affinity matured variants and parental antibodies are shown in Table 1.

TABLE 1
EC50 values of affinity matured variants and
parental antibodies.

| IgG comprising | | EC50 (nM) |
|---|---|---|
| a VH having amino | EC50 | nivolumab |
| acid sequence: | (nM) | analog |
| SEQ ID NO: 1 | 3.81 | 3.47 |
| SEQ ID NO: 2 | 4.49 | |
| SEQ ID NO: 6 | 2.87 | |
| SEQ ID NO: 9 | 5.65 | |
| SEQ ID NO: 5 | 4.91 | 5.79 |
| SEQ ID NO: 4 | 4.12 | |
| SEQ ID NO: 3 | 4.20 | |
| SEQ ID NO: 10 | 11.05 | |

### Example 3—Binding Characteristics

The binding affinity of selected affinity matured PD-1 binding domains was determined using SPR. The binding affinity for human PD-1 was determined in bivalent monospecific IgG format and compared with the binding affinity of bivalent monospecific analogs of reference antibody nivolumab and a bivalent bispecific antibody comprising a binding domain having the sequence of reference antibody nivolumab and a binding domain that binds an unrelated target.

SPR experiments were performed using a Biacore 8K instrument (GE Healthcare) at 25° C. The SPR running buffer (10 mM HEPES, 150 mM NaCl, 3 mM EDTA and 0.05% v/v Surfactant P20, pH 7.4) was prepared from 10X HBS-EP Buffer (GE Healthcare). Anti-human Fc antibodies (GE Healthcare) were immobilized via amine coupling on all sixteen flow cells of an S series sensor chip CM5 (GE Healthcare). The immobilization levels are ˜9000 RU for all flow cells. The desired capturing level (100-150 RU) of anti-PD-1 antibodies was achieved by flowing appropriate concentration of anti-PD-1 antibodies through the active flow cell of each channel for 60 seconds with 10 μL/min flow rate. Then, a PD-1 three-fold serial dilution concentration series (total 7 concentrations, highest at 300 nM) prepared from PD-1 stock (R&D 8986-PD) and running buffer (0 concentration) were injected for 240 seconds (association time) immediately followed by running buffer for 480 seconds (dissociation time) at a flow rate of 45 μL/min. Surface was regenerated with 30-second injection of 3 M MgCl₂ with 30 μL/min flow rate. Binding kinetics and affinity parameters were obtained from a global fit of the data to 1 to 1 binding model.

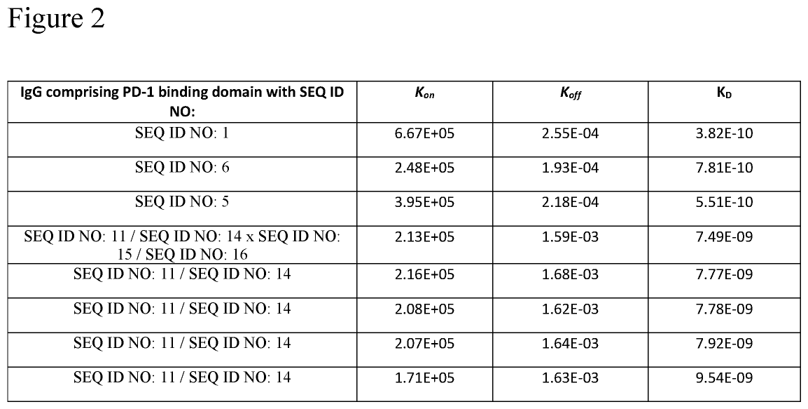

Data is shown in FIG. 2. IgG's comprising the PD-1 binding domains of the present disclosure have an at least ten-fold higher binding affinity (K_{D}) than the analogs of the reference antibody.

Binding affinity was also determined in bispecific IgG format using SPR on a BIAcore-T200 instrument using an anti-huIgG antibody immobilized on a CM5 Series S sensor chip. It was also assessed if the two human proteins can be engaged simultaneously by the bispecific antibodies. The binding affinity of bispecific antibodies comprising a PD-1 binding domain comprising a heavy chain variable region having SEQ ID NO: 7 or a PD-1 binding domain comprising a heavy chain variable region having SEQ ID NO: 8 to human PD-1 and cynomolgus PD-1 was determined. The antibody format used is PD-1×Antigen A, wherein Antigen A is an arbitrarily selected antigen not reactive with PD-1 and not impacting PD-1 binding tested in the assay. Each binding domain of the bispecific antibodies comprises a heavy chain and a light chain. The binding affinity of the bispecific antibodies was compared with the binding affinity of an analog of reference antibody nivolumab, which comprises two anti-PD-1 binding domains.

Reference antibodies used were: nivolumab analog (SEQ ID NO: 13/SEQ ID NO: 14) and a reference antibody against antigen A. An antibody against an unrelated target was used as a negative control for binding. Test antibodies were SEQ ID NO: 7×Antigen A and SEQ ID NO: 8×Antigen A.

Monomeric recombinant antigens used were: hu-Antigen A, cy-Antigen A, huPD-1 (huPD-1-His, Sino Biological, cat. nr. 10377-H08H) and cyPD-1 (cyPD-1-His, R&D Systems, cat. nr. 8509-PD).

### Immobilization

Immobilization of goat anti-huIgG Fc (JIR, cat. nr. 109-005-098) on four flow channels of a CM5 sensor chip (GE Healthcare; Cat. Nr. BR-1005-30) was performed by amine coupling, using 40 μg/ml of the antibody diluted in 10 mM acetate pH 5.0. The following conditions were used: activation time of 420 seconds, deactivation time of 420 seconds, deactivation buffer: 1 M ethanolamine pH 8.5. A high density of immobilization was achieved, ranging from 9158 to 9428 RU.

### Affinity Determination:

For affinity determination, test and control antibodies were captured by anti-huIgG antibody immobilized on the CM5 sensor chip at a flow rate of 30 μl/min for 60 seconds in only one flow cell. Captured antibody concentration was 20 nM for PD-1 affinity determination and 10 nM for Antigen A affinity determination. This was followed by a stabilization period of 60 seconds with buffer at a flow rate of 30 μl/min. Five step, two fold, serial dilutions of the antigens were injected, at 30 μl/min, for 60 seconds, in both the flow cell with the captured antibody and a reference flow cell (no captured antibody). Antigen concentrations were 80 nM down to 2.5 nM for huPD-1 and cyPD-1, and 40 to 1.25 nM for hu-Antigen A and cy-Antigen A. Background correction for buffer effects was performed by injection with buffer alone and the reference flow cell was used for background subtraction.

Following antibody-antigen interaction, an off-rate wash of 300 seconds, at 30 μl/min was done. Regeneration between cycles was done using two 15 μl injections of 10 mM Glycine pH 1.5 at 30 μl/min, followed by a stabilization step of 90 seconds at 90 μl/min. To confirm total regeneration and assay consistency, a repeat run of the reference antibody with all the tested antigen concentrations was performed at the end of the assay and for all antigens tested.

HBS-EP+ buffer was used for PD-1 affinity determination, while, for Antigen A, HBS-EP+ was supplemented with NaCl to a final concentration of 500 mM NaCl, in order to avoid unspecific binding.

Results were analyzed in Biacore T200 Evaluation Software. The raw RU signal were blank subtracted (channel with no captured antibody) and background corrected for buffer effects (subtraction of the run with captured antibody but with buffer in the second injection, instead of antigen). 1:1 binding Langmuir fitting was applied to the set of sample curves, using the simultaneous fitting option of the Biacore T200 Evaluation Software to calculate association rate (ka), dissociation rate (kd) and affinity (KD).

The captured bispecific and reference antibodies showed binding to the respective recombinant antigens. No binding of the antigen to the negative control antibody was observed.

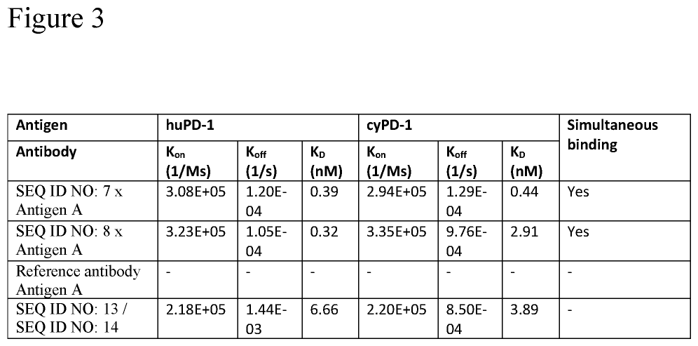

An overview of the data is provided in FIG. 3. For huPD-1, the two bispecific antibodies had similar affinity, both showing more than 10 fold improvement in KD over the reference antibody nivolumab analog, mainly due to the slower dissociation observed. For cyPD-1-His, SEQ ID NO: 7×Antigen A showed approximately 10 fold improvement over nivolumab analog mainly due to the slower dissociation.

### Simultaneous Binding:

Simultaneous binding of the bispecific antibodies to hu-Antigen A and huPD-1 was assayed with a similar set-up as for affinity determination. An immobilized anti-huIgG was used to capture the bispecific antibodies. A mix of nivolumab analog and Antigen A reference antibody was included as a positive control and an antibody against an unrelated target was included as negative control. Then, one of the antigens was injected at a saturating concentration (80 nM for huPD-1 and 40 nM for hu-Antigen A) for 300 sec, to occupy all antigen binding sites. The second antigen was injected sequentially at the same concentration used in injection 1, either alone or in combination with the first antigen (to ensure that all binding sites remained occupied). High salt buffer was used during the whole process, to prevent hu-Antigen A unspecific binding.

SEQUENCES

SEQ ID NO: 1 - Heavy chain variable region - CDRs indicated in bold and underlined
according to Rabat
QVQLQESGPGLVKPSETLSLTCTVSNGSLGFDFWSWIRQPPGRGLEWIGYIYYSGSW
SLNPSFKGRVTMSVDTSKNQFSLNLRSVTAADTAVYYCARGGYTGYGGDWFDPW
GQGTLVTVSS
SEQ ID NO: 2- Heavy chain variable region - CDRs indicated in bold and underlined
according to Kabat
QVQLQESGPGLVKPSETLSLTCTVSNGSLGFEFWSWIRQPPGRGLEWIGYIVYSGSH
SVSPSLKTRVTMSVDTSKNQFSLNLRSVTAADTAVYYCARGGYTGHGGDWFDTW
GQGTLVTVSS
SEQ ID NO: 3- Heavy chain variable region - CDRs indicated in bold and underlined
according to Kabat
QVQLVQSGSELKKPGASVKVSCKASGYTFTRFALSWVRQAPGQGLEWMGWIDPNT
GTPTYAQDFTGRFVFSLDTSVTTAYLQISSLKAEDTAVYYCARSLGYCGSDICYPN
GILDNWGQGTLVTVSS
SEQ ID NO: 4- Heavy chain variable region - CDRs indicated in bold and underlined
according to Kabat
QVQLVQSGSELKKPGASVKVSCKASGYTFTRFAVNWVRQAPGQGLEWMGWIDPN
TGTPTYAQGVTNRFVFSLDTSVTTAYLQISSLKAEDTAVYYCARSLGYCSSDICYP
NLIFDNWGQGTLVTVSS
SEQ ID NO: 5 - Heavy chain variable region - CDRs indicated in bold and underlined
according to Kabat
QVQLVQSGSELKKPGASVKVSCKASGYTFTRFALHWVRQAPGQGLEWMGWIDPN
TGTPTFAQGVTGRFVFSLDTSVTTAYLQISSLKAEDTAVYYCARSLGYCDSDICYP
NWIFDNWGQGTLVTVSS
SEQ ID NO: 6- Heavy chain variable region - CDRs indicated in bold and underlined
according to Kabat
QVQLQESGPGLVKPSETLSLTCTVSDGSIGYHFWSWIRQPPGRGLEWIGYIVYSGSY
NVNPSLKTRVTMSVDTSKNQFSLNLRSVTAADTAVYYCARGGYTGYGGDWFDP
WGQGTLVTVSS
SEQ ID NO: 7- Heavy chain variable region - CDRs indicated in bold and underlined
according to Kabat
QVQLQESGPGLVKPSETLSLTCTVSEGSIGYHFWSWIRQPPGRGLEWIGYIVYSGSY
NVNPSLKTRVTMS
VDTSKNQFSLNLRSVTAADTAVYYCARGGYTGYGGDWFDPWGQGTLVTVSS
SEQ ID NO: 8- Heavy chain variable region - CDRs indicated in bold and underlined
according to Kabat
QVQLVQSGSELKKPGASVKVSCKASGYTFTRFALHWVRQAPGQGLEWMGWIDPN
TGTPTFAQGVTGRFVFSLDTSVTTAYLQISSLKAEDTAVYYCARSLGYCDSDICYP
NWIFDNWGQGTLVTVSS
SEQ ID NO: 9- Heavy chain variable region - CDRs indicated in bold and underlined
according to Kabat
QVQLQESGPGLVKPSETLSLTCTVSNGSLGFYFWSWIRQPPGRGLEWIGYIYYSGST
SFNPSLKSRVTMSVDTSKNQFSLNLRSVTAADTAVYYCARGGYTGYGGDWFDPW
GQGTLVTVSS
SEQ ID NO: 10 - Heavy chain variable region - CDRs indicated in bold and underlined
according to Kabat
QVQLVQSGSELKKPGASVKVSCKASGYTFTRFTMSWVRQAPGQGLEWMGWINPN
TGNPTYAQDFTGRFVFSLDTSVTTAYLQISSLKAEDTAVYYCARILGYCNTDNCYP
NWIFDYWGQGTLVTVSS
SEQ ID NO: 11 - Heavy chain nivolumab analog 1
QVQLVESGGGVVQPGRSLRLDCKASGITFSNSGMHWVRQAPGKGLEWVAVIWYDG
SKRYYADSVKGRFTISRDNSKNTLFLQMNSLRAEDTAVYYCATNDDYWGQGTLVT
VSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKRVEPKSCDKTHTCPPCP
APELGRGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK
SEQ ID NO: 12 - Heavy chain nivolumab analog 2
QVQLVESGGGVVQPGRSLRLDCKASGITFSNSGMHWVRQAPGKGLEWVAVIWYDG
SKRYYADSVKGRFTISRDNSKNTLFLQMNSLRAEDTAVYYCATNDDYWGQGTLVT
VSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPE
FLGGPSVFLFPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK
SEQ ID NO: 13 - Heavy chain nivolumab analog 4
QVQLVESGGGVVQPGRSLRLDCKASGITFSNSGMHWVRQAPGKGLEWVAVIWYDG
SKRYYADSVKGRFTISRDNSKNTLFLQMNSLRAEDTAVYYCATNDDYWGQGTLVT
VSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPE
FLGGPSVFLFPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGS
FFLYSRLWDKSRWQEGNVFSCSVMHEALHNHYTQKSLSLSLGK
SEQ ID NO: 14 - Light chain nivolumab
EIVLTQSPATLSLSPGERATLSCRASQSVSSYLAWYQQKPGQAPRLLIYDASNRATGI
PARFSGSGSGTDFTLTISSLEPEDFAVYYCQQSSNWPRTFGQGTKVEIKRTVAAPSVFI
FPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
SEQ ID NO: 15 - Heavy chain variable region
EVQLVESGGGVVQPGRSLRLSCAASGFTFSNYGMHWVRQAPGKGLEWVAVISYDG
STKYSADSLKGRFTISRDNSKNTLYLQMNSLRADDTAVYYCAKEGWSFDSSGYRSW
FDSWGQGTLVT
SEQ ID NO: 16 - Light chain variable region - CDRs indicated in bold and underlined
according to IMGT
DIQMTQSPSSLSASVGDRVTITCRASQSISSYLNWYQQKPGKAPKLLIYAASSLQSGV
PSRFSGSGSGTDFTLTISSLQPEDFATYYCQQSYSTPPTFGQGTKVEIK
SEQ ID NO: 17-CH1 WT
ASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKRV
SEQ ID NO: 18 - CH2
APELLGGPSVFLFPPKPKDTLMISRTPEVTCVVVDVSHEDPEVKFNWYVDGVEVHNA
KTKPREEQYASTYRVVSVLTVLHQDWLNGKEYKCKVSNKALPAPIEKTISKAK
SEQ ID NO: 19 - CH3
GQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
SEQ ID NO: 20 - Nivolumab analog heavy chain variable region
QVQLVESGGGVVQPGRSLRLDCKASGITFSNSGMHWVRQAPGKGLEWVAVIWYDG
SKRYYADSVKGRFTISRDNSKNTLFLQMNSLRAEDTAVYYCATNDDYWGQGTLVT
VSS
SEQ ID NO: 21 - Nivolumab analog light chain variable region
EIVLTQSPATLSLSPGERATLSCRASQSVSSYLAWYQQKPGQAPRLLIYDASNRATGI
PARFSGSGSGTDFTLTISSLEPEDFAVYYCQQSSNWPRTFGQGTKVEIK
SEQ ID NO: 22 - HCDR1 according to Kabat
FYFWS
SEQ ID NO: 23 - HCDR2 according to Kabat
YIYYSGSTSFNPSLKS
SEQ ID NO: 24 - HCDR3 according to Kabat
GGYTGYGGDWFDP
SEQ ID NO: 25 - HCDR1 according to Kabat
FDFWS
SEQ ID NO: 26 - HCDR2 according to Kabat
YIYYSGSWSLNPSFKG
SEQ ID NO: 27 - HCDR3 according to Kabat
GGYTGYGGDWFDP
SEQ ID NO: 28 - HCDRI according to Kabat
FEFWS
SEQ ID NO: 29 - HCDR2 according to Kabat
YIVYSGSHSVSPSLKT
SEQ ID NO: 30 - HCDR3 according to Kabat
GGYTGHGGDWFDT
SEQ ID NO: 31 - HCDR1 according to Kabat
RFALS
SEQ ID NO: 32 - HCDR2 according to Kabat
WIDPNTGTPTYAQDFTG
SEQ ID NO: 33 - HCDR3 according to Kabat
SLGYCGSDICYPNGILDN
SEQ ID NO: 34 - HCDR1 according to Rabat
RFAVN
SEQ ID NO: 35 - HCDR2 according to Kabat
WIDPNTGTPTYAQGVTN
SEQ ID NO: 36 - HCDR3 according to Kabat
SLGYCSSDICYPNLIFDN
SEQ ID NO: 37 - HCDRI according to Kabat
RFALH
SEQ ID NO: 38 - HCDR2 according to Kabat
WIDPNTGTPTFAQGVTG
SEQ ID NO: 39 - HCDR3 according to Kabat
SLGYCDSDICYPNWIFDN
SEQ ID NO: 40 - HCDR1 according to Kabat
YHFWS
SEQ ID NO: 41 - HCDR2 according to Kabat
YIVYSGSYNVNPSLKT
SEQ ID NO: 42 - HCDR3 according to Kabat
GGYTGYGGDWFDP
SEQ ID NO: 43 - HCDR1 according to Kabat
YHFWS
SEQ ID NO: 44 - HCDR2 according to Kabat
YIVYSGSYNVNPSLKT
SEQ ID NO: 45 - HCDR3 according to Kabat
GGYTGYGGDWFDP
SEQ ID NO: 46 - HCDR1 according to Kabat
RFALH
SEQ ID NO: 47 - HCDR2 according to Kabat.
WIDPNTGTPTFAQGVTG
SEQ ID NO: 48 - HCDR3 according to Kabat
SLGYCDSDICYPNWIFDN
SEQ ID NO: 49 LCDR1 according to IMGT
QSISSY
SEQ ID NO: 50 LCDR2 according to IMGT
AAS
SEQ ID NO: 51 LCDR3 according to IMGT
QQSYSTPPT
SEQ ID NO: 52 Light chain variable region - CDRs indicated in bold and
underlined according to IMGT
DIQMTQSPSSLSASVGDRVTITCRASQSISSYLNWYQQKPGKAPKLLIYAASSLQSGV
PSRFSGSGSGTDFTLTISSLQPEDFATYYCQQSYSTPPITFGQGTRLEIK
SEQ ID NO: 53 Light chain variable region - CDRs indicated in bold and
underlined according to IMGT
EIVMTQSPATLSVSPGERATLSCRASQSVSSNLAWYQQKPGQAPRLLIYGASTRATGI
PARFSGSGSGTEFTLTISSLQSEDFAVYYCQQYNNWPWTFGQGTKVEIK
SEQ ID NO: 54 Light chain variable region - CDRs indicated in bold and
underlined according to IMGT
EIVLTQSPGTLSLSPGERATLSCRASQSVSSSYLAWYQQKPGQAPRLLIYGASSRATGI
PDRFSGSGSGTDFTLTISRLEPEDFAVYYCQQYGSSPWTFGQGTKVEIK
SEQ ID NO: 55 Light chain variable region - CDRs indicated in bold and
underlined according to IMGT
SYVLTQPPSVSVAPGETARITCGGDNIGRKSVYWYQQKSGQAPVLVIYYDSDRPSGI
PERFSGSNSGNTATLTISRVEAGDEADYYCQVWDGSSDHWVFGGGTKLTVL
SEQ ID NO: 56 V region
DIQMTQSPSSLSASVGDRVTITCRASQSISSYLNWYQQKPGKAPKLLIYAASSLQSGV
PSRFSGSGSGTDFTLTISSLQPEDFATYYCQQSYSTP
SEQ ID NO: 57 V region
EIVMTQSPATLSVSPGERATLSCRASQSVSSNLAWYQQKPGQAPRLLIYGASTRATGI
PARFSGSGSGTEFTLTISSLQSEDFAVYYCQQYNNWP
SEQ ID NO: 58 V region
EIVLTQSPGTLSLSPGERATLSCRASQSVSSSYLAWYQQKPGQAPRLLIYGASSRATGI
PDRFSGSGSGTDFTLTISRLEPEDFAVYYCQQYGSSP
SEQ ID NO: 59 V region
SYVLTQPPSVSVAPGETARITCGGDNIGRKSVYWYQQKSGQAPVLVIYYDSDRPSGIP
ERFSGSNSGNTATLTISRVEAGDEADYYCQVWDGSSDH
SEQ ID NO: 60 Light chain constant region
RTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC

1-24. (canceled)

25. A method for treating a disease, comprising administering an effective amount of an anti-human PD-1 binding domain, a binding moiety comprising said anti-PD-1 binding domain, or a pharmaceutical composition comprising said PD-1 binding domain, to an individual in need thereof,
wherein the anti-human PD-1 binding domain has higher binding affinity for human PD-1 than a reference anti-human PD-1 binding domain, wherein the reference anti-human PD-1 binding domain comprises a heavy chain variable region having an amino acid sequence as set forth in SEQ ID NO: 20 and a light chain variable region having an amino acid sequence as set forth in SEQ ID NO: 21.

26. A method for treating a disease associated with a suppressed immune system, in particular for treating cancer, comprising administering an effective amount of an anti-human PD-1 binding domain, a binding moiety comprising said anti-human PD-1 binding domain, or a pharmaceutical composition comprising said anti-human PD-1 binding domain, to an individual in need thereof,
wherein the anti-human PD-1 binding domain has higher binding affinity for human PD-1 than a reference anti-human PD-1 binding domain, wherein the reference anti-human PD-1 binding domain comprises a heavy chain variable region having an amino acid sequence as set forth in SEQ ID NO: 20 and a light chain variable region having an amino acid sequence as set forth in SEQ ID NO: 21.

27. The method for treating a disease according to claim 25, wherein the disease is cancer.

28-37. (canceled)

38. The method for treating a disease according to claim 25, wherein the anti-human PD-1 binding domain comprises at least a heavy chain variable region and a light chain variable region, and wherein the light chain variable region preferably is a light chain variable region of a light chain that is capable of pairing with multiple heavy chains having different epitope specificities.

39. The method for treating a disease according to claim 25, wherein the binding affinity is measured by surface plasmon resonance.

40. The method for treating a disease according to claim 25, wherein the anti-human PD-1 binding domain has at least a ten-fold higher binding affinity for human PD-1 than the reference anti-human PD-1 binding domain.

41. The method for treating a disease according to claim 25, wherein the anti-human PD-1 binding domain has a binding affinity for human PD-1 in a range of about 0.1-1.0 nM, in particular in a range of about 0.3-0.8 nM, more in particular in a range of about 0.38-0.78 nM.

42. The method for treating a disease according to claim 25, wherein the binding affinity is measured with both the anti-human PD-1 binding domain and the reference anti-human PD-1 binding domain in a bivalent monospecific IgG format, or wherein the binding affinity is measured with the anti-human PD-1 binding domain in a bivalent bispecific IgG format and the reference anti-human PD-1 binding domain in a bivalent monospecific IgG format.

43. The method for treating a disease according to claim 25, wherein the anti-human PD-1 binding domain comprises a heavy chain variable region comprising:
a) heavy chain CDR1 (HCDR1), heavy chain CDR2 (HCDR2), and heavy chain CDR3 (HCDR3), having an amino acid sequence as set forth in SEQ ID NO: 22, SEQ ID NO: 23 and SEQ ID NO: 24, respectively;
b) heavy chain CDR1 (HCDR1), heavy chain CDR2 (HCDR2), and heavy chain CDR3 (HCDR3), having an amino acid sequence as set forth in SEQ ID NO: 25, SEQ ID NO: 26, and SEQ ID NO: 27, respectively;
c) heavy chain CDR1 (HCDR1), heavy chain CDR2 (HCDR2), and heavy chain CDR3 (HCDR3), having an amino acid sequence as set forth in SEQ ID NO: 28, SEQ ID NO: 29, and SEQ ID NO: 30, respectively;
d) heavy chain CDR1 (HCDR1), heavy chain CDR2 (HCDR2), and heavy chain CDR3 (HCDR3), having an amino acid sequence as set forth in SEQ ID NO: 31, SEQ ID NO: 32, and SEQ ID NO: 33, respectively;
e) heavy chain CDR1 (HCDR1), heavy chain CDR2 (HCDR2), and heavy chain CDR3 (HCDR3), having an amino acid sequence as set forth in SEQ ID NO: 34, SEQ ID NO: 35, and SEQ ID NO: 36, respectively;
f) heavy chain CDR1 (HCDR1), heavy chain CDR2 (HCDR2), and heavy chain CDR3 (HCDR3), having an amino acid sequence as set forth in SEQ ID NO: 37, SEQ ID NO: 38, and SEQ ID NO: 39, respectively;
g) heavy chain CDR1 (HCDR1), heavy chain CDR2 (HCDR2), and heavy chain CDR3 (HCDR3), having an amino acid sequence as set forth in SEQ ID NO: 40, SEQ ID NO: 41, and SEQ ID NO: 42, respectively;
h) heavy chain CDR1 (HCDR1), heavy chain CDR2 (HCDR2), and heavy chain CDR3 (HCDR3), having an amino acid sequence as set forth in SEQ ID NO: 43, SEQ ID NO: 44, and SEQ ID NO: 45, respectively; or
i) heavy chain CDR1 (HCDR1), heavy chain CDR2 (HCDR2), and heavy chain CDR3 (HCDR3), having an amino acid sequence as set forth in SEQ ID NO: 46, SEQ ID NO: 47, and SEQ ID NO: 48, respectively;
wherein each of the HCDRs may comprise at most three, two, or one amino acid substitution.

44. The method for treating a disease according to claim 43, comprising a heavy chain variable region having an amino acid sequence as set forth in any one of SEQ ID NO: 1-9, or having at least 80%, preferably 85%, more preferably 90%, or most preferably 95% sequence identity thereto.

45. The method for treating a disease according to claim 38, further comprising a CH1 and CL region.

46. A method for treating a disease, comprising administering an effective amount of an anti-human PD-1 binding domain, a binding moiety comprising said anti-PD-1 binding domain, or a pharmaceutical composition comprising said PD-1 binding domain, to an individual in need thereof,
wherein the anti-human PD-1 binding domain comprises a heavy chain variable region comprising:
a) heavy chain CDR1 (HCDR1), heavy chain CDR2 (HCDR2), and heavy chain CDR3 (HCDR3), having an amino acid sequence as set forth in SEQ ID NO: 22, SEQ ID NO: 23 and SEQ ID NO: 24, respectively;
b) heavy chain CDR1 (HCDR1), heavy chain CDR2 (HCDR2), and heavy chain CDR3 (HCDR3), having an amino acid sequence as set forth in SEQ ID NO: 25, SEQ ID NO: 26, and SEQ ID NO: 27, respectively;
c) heavy chain CDR1 (HCDR1), heavy chain CDR2 (HCDR2), and heavy chain CDR3 (HCDR3), having an amino acid sequence as set forth in SEQ ID NO: 28, SEQ ID NO: 29, and SEQ ID NO: 30, respectively;
d) heavy chain CDR1 (HCDR1), heavy chain CDR2 (HCDR2), and heavy chain CDR3 (HCDR3), having an amino acid sequence as set forth in SEQ ID NO: 31, SEQ ID NO: 32, and SEQ ID NO: 33, respectively;
e) heavy chain CDR1 (HCDR1), heavy chain CDR2 (HCDR2), and heavy chain CDR3 (HCDR3), having an amino acid sequence as set forth in SEQ ID NO: 34, SEQ ID NO: 35, and SEQ ID NO: 36, respectively;
f) heavy chain CDR1 (HCDR1), heavy chain CDR2 (HCDR2), and heavy chain CDR3 (HCDR3), having an amino acid sequence as set forth in SEQ ID NO: 37, SEQ ID NO: 38, and SEQ ID NO: 39, respectively;
g) heavy chain CDR1 (HCDR1), heavy chain CDR2 (HCDR2), and heavy chain CDR3 (HCDR3), having an amino acid sequence as set forth in SEQ ID NO: 40, SEQ ID NO: 41, and SEQ ID NO: 42, respectively;
h) heavy chain CDR1 (HCDR1), heavy chain CDR2 (HCDR2), and heavy chain CDR3 (HCDR3), having an amino acid sequence as set forth in SEQ ID NO: 43, SEQ ID NO: 44, and SEQ ID NO: 45, respectively; or
i) heavy chain CDR1 (HCDR1), heavy chain CDR2 (HCDR2), and heavy chain CDR3 (HCDR3), having an amino acid sequence as set forth in SEQ ID NO: 46, SEQ ID NO: 47, and SEQ ID NO: 48, respectively;
wherein each of the HCDRs may comprise at most three, two, or one amino acid substitution.

47. The method for treating a disease according to claims 46, comprising a heavy chain variable region having an amino acid sequence as set forth in any one of SEQ ID NO: 1-9, or having at least 80%, preferably 85%, more preferably 90%, or most preferably 95% sequence identity thereto.

48. A method for treating a disease, comprising administering an effective amount of an anti-human PD-1 binding domain, a binding moiety comprising said anti-PD-1 binding domain, or a pharmaceutical composition comprising said PD-1 binding domain, to an individual in need thereof,
wherein the anti-human PD-1 binding domain when monovalently present in a bivalent antibody, provides comparable, or equal or higher, potency in blocking ligand binding to PD-1 than a reference anti-human PD-1 antibody, wherein the reference anti-human PD-1 antibody comprises two heavy chain variable regions having an amino acid sequence as set forth in SEQ ID NO: 20 and two light chain variable regions having an amino acid sequence as set forth in SEQ ID NO: 21.

49. The method for treating a disease according to claim 48, wherein the potency in blocking ligand binding to PD-1 is measured in a PD-1/PD-L1 reporter assay.

50. The method for treating a disease according to claim 48, wherein a comparable potency in blocking ligand binding to PD-1 activity is a potency within a 5 fold range of the potency in blocking ligand binding to PD-1 of the reference anti-human PD-1 antibody.

51. The method for treating a disease according to claim 50, wherein a comparable potency in blocking ligand binding to PD-1 activity includes a 5, 4, 3, and 2 fold deviation from the potency in blocking ligand binding to PD-1 of the reference anti-human PD-1 antibody.


SEQUENCE LISTING (66 sequences)

SEQ ID NO: 1 - protein, 121 aa, synthetic construct
  REGION 1..121: note = Heavy chain variable region
QVQLQESGPGLVKPSETLSLTCTVSNGSLGFDFWSWIRQPPGRGLEWIGYIYYSGSWSLNPSFKGRVTMSVDTSKNQFSLNLRSVTAADTAVYYCARGGYTGYGGDWFDPWGQGTLVTVSS

SEQ ID NO: 2 - protein, 121 aa, synthetic construct
  REGION 1..121: note = Heavy chain variable region
QVQLQESGPGLVKPSETLSLTCTVSNGSLGFEFWSWIRQPPGRGLEWIGYIVYSGSHSVSPSLKTRVTMSVDTSKNQFSLNLRSVTAADTAVYYCARGGYTGHGGDWFDTWGQGTLVTVSS

SEQ ID NO: 3 - protein, 127 aa, synthetic construct
  REGION 1..127: note = Heavy chain variable region
QVQLVQSGSELKKPGASVKVSCKASGYTFTRFALSWVRQAPGQGLEWMGWIDPNTGTPTYAQDFTGRFVFSLDTSVTTAYLQISSLKAEDTAVYYCARSLGYCGSDICYPNGILDNWGQGTLVTVSS

SEQ ID NO: 4 - protein, 127 aa, synthetic construct
  REGION 1..127: note = Heavy chain variable region
QVQLVQSGSELKKPGASVKVSCKASGYTFTRFAVNWVRQAPGQGLEWMGWIDPNTGTPTYAQGVTNRFVFSLDTSVTTAYLQISSLKAEDTAVYYCARSLGYCSSDICYPNLIFDNWGQGTLVTVSS

SEQ ID NO: 5 - protein, 127 aa, synthetic construct
  REGION 1..127: note = Heavy chain variable region
QVQLVQSGSELKKPGASVKVSCKASGYTFTRFALHWVRQAPGQGLEWMGWIDPNTGTPTFAQGVTGRFVFSLDTSVTTAYLQISSLKAEDTAVYYCARSLGYCDSDICYPNWIFDNWGQGTLVTVSS

SEQ ID NO: 6 - protein, 121 aa, synthetic construct
  REGION 1..121: note = Heavy chain variable region
QVQLQESGPGLVKPSETLSLTCTVSDGSIGYHFWSWIRQPPGRGLEWIGYIVYSGSYNVNPSLKTRVTMSVDTSKNQFSLNLRSVTAADTAVYYCARGGYTGYGGDWFDPWGQGTLVTVSS

SEQ ID NO: 7 - protein, 121 aa, synthetic construct
  REGION 1..121: note = Heavy chain variable region
QVQLQESGPGLVKPSETLSLTCTVSEGSIGYHFWSWIRQPPGRGLEWIGYIVYSGSYNVNPSLKTRVTMSVDTSKNQFSLNLRSVTAADTAVYYCARGGYTGYGGDWFDPWGQGTLVTVSS

SEQ ID NO: 8 - protein, 127 aa, synthetic construct; identical to SEQ ID NO: 5
  REGION 1..127: note = Heavy chain variable region

SEQ ID NO: 9 - protein, 121 aa, synthetic construct
  REGION 1..121: note = Heavy chain variable region
QVQLQESGPGLVKPSETLSLTCTVSNGSLGFYFWSWIRQPPGRGLEWIGYIYYSGSTSFNPSLKSRVTMSVDTSKNQFSLNLRSVTAADTAVYYCARGGYTGYGGDWFDPWGQGTLVTVSS

SEQ ID NO: 10 - protein, 127 aa, synthetic construct
  REGION 1..127: note = Heavy chain variable region
QVQLVQSGSELKKPGASVKVSCKASGYTFTRFTMSWVRQAPGQGLEWMGWINPNTGNPTYAQDFTGRFVFSLDTSVTTAYLQISSLKAEDTAVYYCARILGYCNTDNCYPNWIFDYWGQGTLVTVSS

SEQ ID NO: 11 - protein, 443 aa, synthetic construct
  REGION 1..443: note = Heavy chain Nivolumab analog 1
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 440 aa, synthetic construct
  REGION 1..440: note = Heavy chain Nivolumab analog 2
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 440 aa, synthetic construct; identical to SEQ ID NO: 12
  REGION 1..440: note = Heavy chain Nivolumab analog 4

SEQ ID NO: 14 - protein, 214 aa, synthetic construct
  REGION 1..214: note = Light chain Nivolumab
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 122 aa, synthetic construct
  REGION 1..122: note = Heavy chain variable region
EVQLVESGGGVVQPGRSLRLSCAASGFTFSNYGMHWVRQAPGKGLEWVAVISYDGSTKYSADSLKGRFTISRDNSKNTLYLQMNSLRADDTAVYYCAKEGWSFDSSGYRSWFDSWGQGTLVT

SEQ ID NO: 16 - protein, 107 aa, synthetic construct
  REGION 1..107: note = Light chain variable region
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 17 - protein, 98 aa, synthetic construct
  REGION 1..98: note = CH1 WT
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 18 - protein, 110 aa, synthetic construct
  REGION 1..110: note = CH2
APELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAK

SEQ ID NO: 19 - protein, 107 aa, synthetic construct
  REGION 1..107: note = CH3
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 113 aa, synthetic construct [sequence omitted]

SEQ ID NO: 21 - protein, 107 aa, synthetic construct [sequence omitted]

SEQ ID NO: 22 - protein, 5 aa, synthetic construct
  REGION 1..5: note = HCDR1
FYFWS

SEQ ID NO: 23 - protein, 16 aa, synthetic construct
  REGION 1..16: note = HCDR2
YIYYSGSTSFNPSLKS

SEQ ID NO: 24 - protein, 13 aa, synthetic construct
  REGION 1..13: note = HCDR3
GGYTGYGGDWFDP

SEQ ID NO: 25 - protein, 5 aa, synthetic construct
  REGION 1..5: note = HCDR1
FDFWS

SEQ ID NO: 26 - protein, 16 aa, synthetic construct
  REGION 1..16: note = HCDR2
YIYYSGSWSLNPSFKG

SEQ ID NO: 27 - protein, 13 aa, synthetic construct
  REGION 1..13: note = HCDR3
GGYTGYGGDWFDP

SEQ ID NO: 28 - protein, 5 aa, synthetic construct
  REGION 1..5: note = HCDR1
FEFWS

SEQ ID NO: 29 - protein, 16 aa, synthetic construct
  REGION 1..16: note = HCDR2
YIVYSGSHSVSPSLKT

SEQ ID NO: 30 - protein, 13 aa, synthetic construct
  REGION 1..13: note = HCDR3
GGYTGHGGDWFDT

SEQ ID NO: 31 - protein, 5 aa, synthetic construct
  REGION 1..5: note = HCDR1
RFALS

SEQ ID NO: 32 - protein, 17 aa, synthetic construct
  REGION 1..17: note = HCDR2
WIDPNTGTPTYAQDFTG

SEQ ID NO: 33 - protein, 18 aa, synthetic construct
  REGION 1..18: note = HCDR3
SLGYCGSDICYPNGILDN

SEQ ID NO: 34 - protein, 5 aa, synthetic construct
  REGION 1..5: note = HCDR1
RFAVN

SEQ ID NO: 35 - protein, 17 aa, synthetic construct
  REGION 1..17: note = HCDR2
WIDPNTGTPTYAQGVTN

SEQ ID NO: 36 - protein, 18 aa, synthetic construct
  REGION 1..18: note = HCDR3
SLGYCSSDICYPNLIFDN

SEQ ID NO: 37 - protein, 5 aa, synthetic construct
  REGION 1..5: note = HCDR1
RFALH

SEQ ID NO: 38 - protein, 17 aa, synthetic construct
  REGION 1..17: note = HCDR2
WIDPNTGTPTFAQGVTG

SEQ ID NO: 39 - protein, 18 aa, synthetic construct
  REGION 1..18: note = HCDR3
SLGYCDSDICYPNWIFDN

SEQ ID NO: 40 - protein, 5 aa, synthetic construct
  REGION 1..5: note = HCDR
YHFWS

SEQ ID NO: 41 - protein, 16 aa, synthetic construct
  REGION 1..16: note = HCDR2
YIVYSGSYNVNPSLKT

SEQ ID NO: 42 - protein, 13 aa, synthetic construct
  REGION 1..13: note = HCDR3
GGYTGYGGDWFDP

SEQ ID NO: 43 - protein, 5 aa, synthetic construct
  REGION 1..5: note = HCDR1
YHFWS

SEQ ID NO: 44 - protein, 16 aa, synthetic construct
  REGION 1..16: note = HCDR2
YIVYSGSYNVNPSLKT

SEQ ID NO: 45 - protein, 13 aa, synthetic construct
  REGION 1..13: note = HCDR3
GGYTGYGGDWFDP

SEQ ID NO: 46 - protein, 5 aa, synthetic construct
  REGION 1..5: note = HCDR1
RFALH

SEQ ID NO: 47 - protein, 17 aa, synthetic construct
  REGION 1..17: note = HCDR2
WIDPNTGTPTFAQGVTG

SEQ ID NO: 48 - protein, 18 aa, synthetic construct
  REGION 1..18: note = HCDR3
SLGYCDSDICYPNWIFDN

SEQ ID NO: 49 - protein, 6 aa, synthetic construct
  REGION 1..6: note = LCDR1
QSISSY

SEQ ID NO: 50 - skipped

SEQ ID NO: 51 - protein, 9 aa, synthetic construct
  REGION 1..9: note = LCDR3
QQSYSTPPT

SEQ ID NO: 52 - protein, 108 aa, synthetic construct
  REGION 1..108: note = Light chain variable region
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 53 - protein, 107 aa, synthetic construct
  REGION 1..107: note = Light chain variable region
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 54 - protein, 108 aa, synthetic construct
  REGION 1..108: note = Light chain variable region
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 55 - protein, 108 aa, synthetic construct
  REGION 1..108: note = Light chain variable region
SYVLTQPPSVSVAPGETARITCGGDNIGRKSVYWYQQKSGQAPVLVIYYDSDRPSGIPERFSGSNSGNTATLTISRVEAGDEADYYCQVWDGSSDHWVFGGGTKLTVL

SEQ ID NO: 56 - protein, 95 aa, synthetic construct
  REGION 1..95: note = V region
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 57 - protein, 95 aa, synthetic construct
  REGION 1..95: note = V region
EIVMTQSPATLSVSPGERATLSCRASQSVSSNLAWYQQKPGQAPRLLIYGASTRATGIPARFSGSGSGTEFTLTISSLQSEDFAVYYCQQYNNWP

SEQ ID NO: 58 - protein, 96 aa, synthetic construct
  REGION 1..96: note = V region
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 59 - protein, 96 aa, synthetic construct
  REGION 1..96: note = V region
SYVLTQPPSVSVAPGETARITCGGDNIGRKSVYWYQQKSGQAPVLVIYYDSDRPSGIPERFSGSNSGNTATLTISRVEAGDEADYYCQVWDGSSDH

SEQ ID NO: 60 - protein, 107 aa, synthetic construct [sequence omitted]

SEQ ID NO: 61 - skipped

SEQ ID NO: 62 - protein, 16 aa, synthetic construct
  REGION 1..16: note = HCDR2
  VARIANT 3: note = can be Y, V, or I
  VARIANT 7: note = can be S, or G
  VARIANT 8: note = can be T, Y, S, H, N, W, L, or Q
  VARIANT 9: note = can be S, or N
  VARIANT 10: note = can be F, V, or L
  VARIANT 11: note = can be N, or S
  VARIANT 13: note = can be S or A
  VARIANT 14: note = can be F or L
  VARIANT 16: note = can be S, T, G, D, R, or N
YIXYSGXXXXXPXXKX

SEQ ID NO: 63 - protein, 13 aa, synthetic construct
  REGION 1..13: note = HCDR3
  VARIANT 6: note = can be Y, H, V, or A
  VARIANT 13: note = can be P, V, Y, W, F, T, Q, H, or S
GGYTGXGGDWFDX

SEQ ID NO: 64-65 - skipped (2 entries)

SEQ ID NO: 66 - protein, 18 aa, synthetic construct
  REGION 1..18: note = HCDR3
  VARIANT 1: note = can be I, S, or V
  VARIANT 2: note = can be L, Q, or N
  VARIANT 6: note = can be N, G, S, or D
  VARIANT 7: note = can be T, S, P, N, or E
  VARIANT 9: note = can be N, or I
  VARIANT 14: note = can be W, G, Q, H, W, A, or L
  VARIANT 15: note = can be I, V, or L
  VARIANT 16: note = can be F, L, or I
  VARIANT 18: note = can be Y, S, N, I, R, H, V, T, K, A, or L
XXGYCXXDXCYPNXXXDX